SEQUENCE LISTING (1 sequence)

SEQ ID NO: 1 - protein, 17 aa, Bacillus thuringiensis
WTCWSCLVCAACSVELL

(19) United States
(12) Patent Application Publication
(10) Pub. No.: US 2008/0248953 A1
(43) Pub. Date: Oct. 9, 2008

(54) Use of Bacteriocins For Promoting Plant Growth and Disease Resistance

(75) Inventors: Donald Smith, Sainte-Anne-de-Bellevue (CA); Kyung Dong Lee, Sainte-Anne-de-Bellevue (CA); Elizabeth Gray, Montreal (CA); Alfred Souleimanov, Montreal (CA); Xioamin Zhou, Sainte-Anne-de-Bellevue (CA)

Correspondence Address: DRINKER BIDDLE & REATH (DC), 1500 K STREET, N.W., SUITE 1100, WASHINGTON, DC, 20005-1209, US

(73) Assignee: MCGILL UNIVERSITY

(21) Appl. No.: 12/093,779

(22) PCT Filed: Nov. 15, 2006
(86) PCT No.: PCT/CA2006/001861

§ 371 Date: May 16, 2008

Related U.S. Application Data
(60) Provisional application No. 60/737,404, filed on Nov. 17, 2005.

(51) Int. Cl. A01N 63/02; C07K 14/32; C12N 15/31; C12N 1/21; A01N 25/26; G01N 33/50; A01P 21/00; A01P 1/00

(52) U.S. Cl. 504/100; 504/117; 530/350; 536/23.7; 435/252.31; 436/86

(57) ABSTRACT

A method for promoting plant growth and/or disease resistance comprising applying a purified polypeptide that is a bacteriocin and that possesses plant growth and/or disease resistance promoting activity to a plant or plant seed, or to the growing environment thereof.

### CROSS-REFERENCE TO RELATED APPLICATIONS

This application claims the benefit of and priority from U.S. Provisional Patent Application No. 60/737,404 filed Nov. 17, 2005, which is incorporated by reference herein.

### FIELD OF THE INVENTION

This invention relates to purified polypeptides that are bacteriocins and that possess plant growth and/or disease resistance promoting activity, and their use in e.g. promoting plant growth, promoting disease resistance in plants, and as bactericidal or bacteristatic agents.

### BACKGROUND OF THE INVENTION

Bacteriocins are proteins produced by prokaryotes that are bactericidal and/or bacteristatic against organisms related to the producer strain, but that do not act against the producer strain itself.

Bacteria-produced compounds of various kinds are known to have plant growth promoting activity. For instance lipo-chitooligosaccharides (LCOs) or nodulation (NOD) factors, produced by certain rhizobia, have been demonstrated to increase plant germination.

However, compounds known to improve plant growth at low concentrations have not been proteins produced by other organisms. Until the instant invention, there have not been reports of bacteriocins that increase plant growth or disease resistance.

### SUMMARY OF THE INVENTION

The inventors have discovered, surprisingly, that bacteriocins may be used to promote plant growth and/or promote disease resistance in plants.

Accordingly, in one aspect, the invention provides a method for promoting plant growth and/or disease resistance comprising applying a purified polypeptide that is a bacteriocin and that possesses plant growth and/or disease resistance promoting activity to a plant or plant seed, or in the growing environment thereof.

In another aspect, the invention provides a purified polypeptide that is a bacteriocin and that possesses plant growth and/or disease resistance promoting activity, said polypeptide being selected from the group consisting of:

(a) a polypeptide comprising the partial amino acid sequence

| | |
|---|---|
| WTCWSCLVCAACSVELL; | (SEQ ID NO: 1) |

(b) a polypeptide possessing the bacteriocin and plant growth and/or disease resistance promoting activities of the polypeptide of (a), and which comprises a sequence of 17 contiguous amino acids possessing at least 70% sequence identity to SEQ ID NO: 1; and

(c) a polypeptide which is a fragment of the polypeptide of (a) or (b), said fragment possessing the bacteriocin and plant growth and/or disease resistance promoting activities of the polypeptide of (a).

In another aspect, the invention provides a composition comprising a purified polypeptide as described above, and a carrier or diluent.

In another aspect, the invention provides an isolated polynucleotide encoding a polypeptide as described above, or the complement thereto.

In another aspect, the invention provides a vector comprising a polynucleotide or host cell as described above.

In another aspect, the invention provides a method for producing a polypeptide comprising culturing the host cell as described above under conditions sufficient for expression of the polypeptide encoded by said polynucleotide, and recovering said polypeptide.

In another aspect, the invention provides a plant growth and/or disease resistance promoting composition comprising a purified polypeptide that is a bacteriocin and that possesses plant growth and/or disease resistance promoting activity, and a carrier or diluent.

In another aspect, the invention provides a plant seed treated with the plant growth and/or disease resistance promoting composition as described above.

In another aspect, the invention provides a kit comprising a plant growth and/or disease resistance promoting composition as described above and instructions for use.

In another aspect, the invention provides a method for obtaining a polypeptide that is a bacteriocin and that possesses plant growth and/or disease resistance promoting activity comprising:

(a) providing a polypeptide;

(b) determining whether said polypeptide promotes plant growth and/or disease resistance; and

(c) determining whether said polypeptide has bactericidal and/or bacteristatic properties.

In another aspect, the invention provides a method for obtaining a polypeptide that is a bacteriocin and that possesses plant growth and/or disease resistance promoting activity, comprising:

(a) providing a bacteriocin; and

(b) determining whether said bacteriocin has plant growth and/or disease resistance promoting properties.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1A-C illustrate HPLC analysis of the three samples: (A) PPBP, Partially Purified Bacterial Peptide, prepared by HPLC purification; (B) medium control, exposed to the exact same conditions as PPBP, including butanol extraction, HPLC purification; (C)CFS, Cell Free Supernatant, prepared by differential centrifugation of the bacterial culture.

FIG. 2A-C illustrate the bactericidal and/or bacteristatic effects on Bacillus thuringiensis NEB 17 (A), Bacillus cereus ATCC 14579 (B) and Bacillus thuringiensis ssp thuringiensis Bt1627 (C) exposed to 0 μL (circles), 100 μL (closed squares), 300 μL (triangles), and 600 μL (open squares) of PPBP (0.066 μg μl⁻¹).

FIG. 3 illustrates a SDS-PAGE analysis on PPBP and the CFS, as well as direct detection of PPBP and CFS. 20 μL of PPBP and CFS were loaded into wells, media exposed to the same conditions as for the PPBP and CFS served as controls. For direct detection of bacteriocin activity, 35 μL of PPBP and CFS were loaded into wells, and the respective media control was also used. The gel, overlaid with a soft agar King's medium, was inoculated with the indicator strain, Bacillus thuringiensis ssp. thuringiensis Bt1627. Lane 1: low molecular weight marker (MKR); Lane 2: loading dye control (LD), Lane 3: CFS; Lane 4: PPBP; Lane 5: centrifuged media control (CM ctl); Lane 6: purified media control (PM ctl); Lane 7: PPBP for direct detection; Lane 8: CFS for direct detection; Lane 9: purified media control (PM ctl) and Lane 10: centrifuged media control (CM ctl).

FIG. 4 illustrates MALDI-QTOF (Matrix Assisted Laser Desorption Ionization—Quadrapole Time of Flight) mass spectrometry analysis of the PPBP, partially purified via reversed phase HPLC, and collected in 60% acetylnitrile.

FIG. 5 illustrates MALDI-QTOF mass spectrometry of partially purified thuricin 17 (PPT17). Thuricin 17 was partially purified via reverse phase HPLC, and collected in 60% acetonitrile. Sequence analysis via Edman degradation was determined and the presence of cysteines was detected via ms/ms fragment analysis of the parent ion. Analysis was conducted on two separate biological replicates that were grown and extracted separately; similar results were obtained from each.

FIG. 6A-C illustrate a visual representation of inhibition of thuricin 17 as it relates to its production. Age of culture, via optical density, was initially determined via spectrophotometry, (A) 1.46; (B) 1.30 and (C) 1.13. Inhibition by thuricin 17 was conducted via the disk diffusion assay on the indicator strain Bacillus thuringiensis ssp.

FIG. 7 illustrates thuricin 17 production by Bacillus thuringiensis NEB 17 over time. Sample aliquots were removed at hourly intervals and the O.D. ₆₀₀ₙₘ recorded. In parallel, aliquots were diluted to determine the viable cell count (CFU). Production of thuricin 17 was quantified into activity units (AU) by preparing a series of two-fold dilutions and testing against the indicator strain B. thuringiensis ssp. thuringiensis Bt 1627.

FIG. 8A-C illustrate HPLC analysis of (A) the crude extract from Bacillus thuringiensis NEB17; (B) partially purified thuricin 17, and (C) King's Medium B without bacteria, as a control.

FIG. 9 illustrates the bacteriocin effects of thuricin 17. Controls were the producer strain, Bacillus thuringiensis NEB 17 (A), as well as purified media without thuricin 17 tested on B. cereus ATCC 14579 (B). Strains showing inhibition are B. cereus ATCC 14579 (C), and Brevibacillus brevis ATCC 8246 (D).

FIG. 10A-C illustrates the characterization of the plant biological activity of thuricin 17 on soybean (Glycine max L.) germination (%). The chromatogram was initially separated into 5 fractions (61-70, 71-80, 81-90, 91-100 and 101-110 minutes) (FIG. 10A), then further subdivided (B) 81-82, 83-84, 85-86, 87-88 and 89-90 (FIG. 10B). Material from B, thuricin 17 purified from fraction 86-87) was then assessed to determine the optimum concentration for increasing germination, (FIG. 10C). Bars represent±SE (n=10).

FIG. 11A-D illustrates HPLC chromatograms of the entire extract of Bacillus thuringiensis NEB17 before the purification (A), and compounds eluted with 35% acetonitrile (B), 43% acetonitrile (C) and 100% acetonitrile (D).

FIG. 12 illustrates a schematic diagram of planting methodology for corn seeds supplied with varied concentrations of thuricin 17 solutions.

FIG. 13 illustrates corn seedling emergence (%) at 72 h, 76 h, 80 h and 84 h after eight treatments with one of three different solutions of thuricin 17 (10⁻⁹ M, 10⁻¹⁰ M, or 10⁻¹¹ M) or a control treatment. Values are means ±SE of n=4-5 replicates.

FIG. 14 illustrates tomato seedling emergence (%) at 72 h, 120 h, 144 h and 168 h after eight treatment with one of three different solutions of thuricin 17 (10⁻⁹ M, 10⁻¹⁰ M, or 10⁻¹¹ M) or control treatments. Values are means ±SE of n=4-5 replicates.

FIG. 15A-B illustrate soybean leaf area (FIG. 15A) and shoot dry weights (FIG. 15B) at 14 days after treatment with the bacteriocin extracted from Bacillus cereus UW85 (cerecin 85) at 10⁻⁹ M, 10⁻¹¹M, or 10⁻¹¹ M.

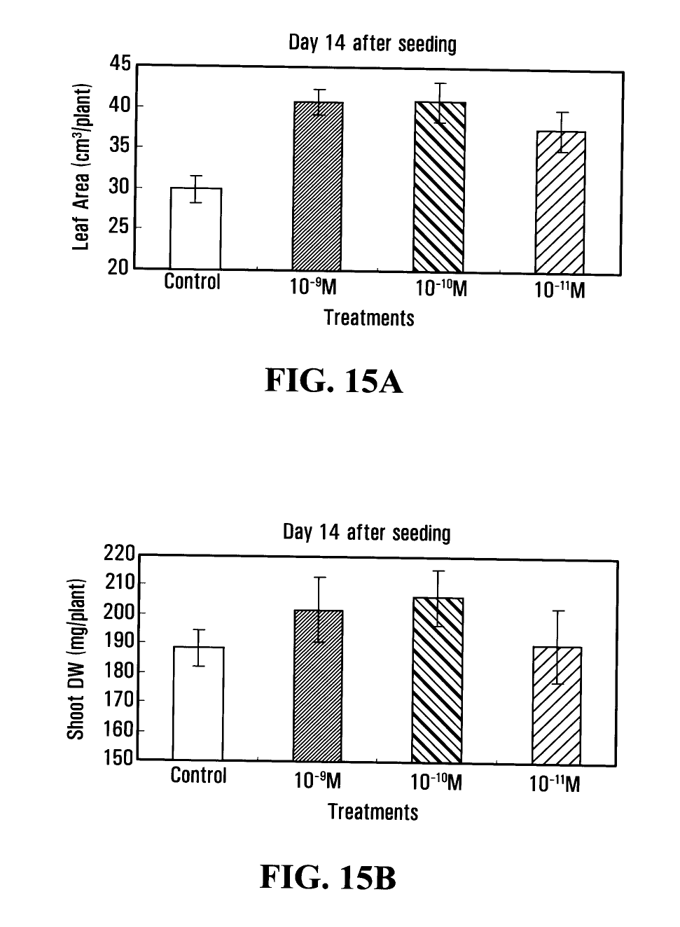

FIG. 16A-B illustrate changes in phenylalanine ammonia lyase (PAL) (FIG. 16A) and tyrosine ammonia lyase (TAL) (FIG. 16B) activities in soybean leaves after treatment with chitin hexamer (0.5 ml (100 μmol/L)) and thuricin 17 (1×10⁸ M). Control (open circles), chitin hexamer [(GlcNAc)₆] (circles), thuricin 17 (triangles), chitin hexamer and thuricin 17 (squares). Each point represents the mean ±SE (n=3).

FIG. 17 illustrates changes of total phenolics in soybean leaves after treatment with chitin hexamer and thuricin 17. T0: control; T1: chitin hexamer [(GlcNAc)₆], T2: T17; T3: chitin hexamer and thuricin 17. Each bar represents the mean ±SE (n=3).

FIG. 18A-B illustrate changes of peroxidase (FIG. 18A) and superoxide dismutase (FIG. 18B) activities in soybean leaves after treatment with chitin hexamer and thuricin 17. T0: control; T1: chitin hexarner [(GlcNAc)₆]; T2: thuricin 17; T3: chitin hexamer and thuricin 17. Each bar represents the mean ±SE (n=3).

FIG. 19A-C illustrate active staining of peroxidase (POD) (FIG. 19A), catalase (CAT) (FIG. 19B) and superoxide dismutase (SOD) (FIG. 19C) in soybean leaves after treatment with chitin hexamer and thuricn 17 ((a) PAGE; (b) inactivated by H₂O₂; and (c) inactivated by KCN). T0: control; T1: chitin hexamer [(GlcNAc)₆]; T2: thuricin 17, T3: chitin hexamer and thuricin 17.

### DETAILED DESCRIPTION

The invention provides a method for promoting plant growth and/or disease resistance comprising applying a purified polypeptide that is a bacteriocin and that possesses plant growth and/or disease resistance promoting activity to a plant or plant seed, or in the growing environment thereof. The polypeptides used in the methods of the invention exhibit at least one plant growth and/or disease resistance promoting property and also have at least one property of a bacteriocin. Specifically, the polypeptides demonstrate at least one bactericidal or bacteristatic activity against a related or unrelated bacterial strain, preferably a related strain.

As used herein, the term “polypeptide” encompasses any chain of naturally or non-naturally occurring amino acids (either D)- or L-amino acids), regardless of length (e.g., at least 5, 6, 8, 10, 12, 14, 16, 18, 20, 25, 30, 40, 50, 100 or more amino acids) or post-translational modification (e.g., glycosylation or phosphorylation) or the presence of e.g. one or more non-amino acyl groups (for example, sugar, lipid, etc.) covalently linked to the peptide, and includes, for example, natural proteins, synthetic or recombinant polypeptides and peptides, hybrid molecules, peptoids, peptidomimetics, etc.

As used herein, the term “bacteriocin” means a protein or peptide produced by a prokaryote (typically a Gram-negative or Gram-positive bacterium) and that is bactericidal and/or bacteristatic against organisms related to the producer strain, but that does not act against the producer strain itself. Many but not all bacteriocins are of low-molecular weight, in the range of about 100 to about 10,000 Daltons. Bacteriocins are known to inhibit growth of closely related microorganisms thereby eliminating or significantly reducing competition for available nutrients (Jack et al. Microbiol. Rev., 59:171-200, 1995). Bacteriocins have also been implicated as playing a role as antibiotics against pathogenic bacteria and as natural food preservatives.

As used herein, the term “plant growth promoting activity” encompasses a wide range of improved plant properties, including, without limitation, improved nodulation (e.g. increased number of nodules), nitrogen fixation (e.g. increased nitrogen concentration as measured by mg g⁻¹ dry weight of plant material), increased leaf area, increased seed germination, increased leaf greenness (e.g. as measured by SPAD), increased photosynthesis (μmol cm⁻² s⁻¹), or an increase in accumulated dry-weight of the plant.

As used herein, the term “plant disease resistance promoting activity” or the like, encompasses, without limitation, increased resistance to pathogen attack or increased production of one or more secondary metabolites that function to improve the resistance of a plant to pathogen attack, as discussed herein.

Polypeptides useful in practicing the methods of the invention can be obtained in a number of ways. For example, any polypeptide of interest may be screened, either sequentially in either order, or simultaneously, for a plant growth and/or disease resistance promoting activity and for activity as a bacteriocin. In one embodiment, the polypeptide will be produced by a bacterial strain known to be a plant growth promoting strain such as a PGPR. In another embodiment, the polypeptide is obtained from a bacterial strain and known to be a producer of bacteriocin.

Methods for testing compounds for bactericidal or bacteristatic properties are known in the art. For example, a zone of inhibition assay such as an agar disc diffusion assay may be used to test the polypeptides of interest or bactericidal or bacteristatic activity against various indicator strains.

Assessment of the plant growth promoting activity of polypeptides may be accomplished by known methods. For instance, a polypeptide of interest may be applied by leaf spray or root irrigation to test plants, such as soybean plants. Plants may then be grown under controlled environment conditions (growth chamber or greenhouse) for e.g. about 40 days. At harvest, data may be collected concerning e.g. plant height, leaf greenness, leaf area, nodule number, nodule dry weight, shoot and dry root weight or length, nitrogen content and photosynthesis and compared to controls.

Assessment of plant disease resistance promoting activity of polypeptides may also be accomplished by known methods, such as by detecting or measuring a reduction in pathogen infestation of a plant, or indirectly by detecting or measuring increased production of one or more secondary metabolites that function to improve the resistance of a plant to pathogen attack. Exemplary secondary metabolites include lignification-related enzymes such as phenylalanine ammonia lyase (PAL), and tyrosine ammonia lyase (TAL), antioxidative enzymes such as peroxidase (POD), catalase (CAT), and superoxidase dismutase (SOD), and total phenolic compounds. Various methods for detecting or measuring increases in enzyme activity levels in plants (e.g. PAL, TAL, POD, CAD and SOD) are known in the art and exemplary techniques are described in the examples herein. Similarly, techniques for determining concentrations or levels of total phenolic compounds are known and exemplary methods are described in the examples herein.

An increase or improvement in plant growth or disease resistance means a statistically significant increase or improvement in the measured criterion of plant growth or disease resistance in a plant treated with a polypeptide according to the invention relative to an untreated control plant.

Bacteria that are known to produce bacteriocins include, but are not limited to, Bacillus, Pseudomonas, Rhizobium, Braydyrhizobium and Lactoccus species.

Depending on their structure, mode of action and chemical properties, four distinct classes of bacteriocins are recognized (Klaenhammer 1993). Current classifications of bacteriocins include Class I-type A lantibiotics, Class I-type B lantibiotics, Class IIa, Class IIb, Class IIc and Class III (Eijsink et al. 2002; Chen and Hoover 2003). Nisin, for example, is a widely characterized bacteriocin produced from the lactic acid bacterium, Lactococcus lactis, and has been accepted by the World Health Organization (WHO) as a food biopreservative (Hansen 1994). Current applications of bacteriocins are as food preservatives while less research has been conducted on the agricultural applications of bacteriocins.

Most known bacteriocin producing Bacillus species are from either soil or food isolates. B. thuringiensis HD2 synthesizes thuricin HD2, 950 kDa (Favret and Yousten 1989). Thuricin 7, 11.6 kDa, is produced by a soil isolate, B. thuringiensis BMG1.7 (Cherif et al. 2001). B. thuringiensis ssp. tochigiensis HD868 produces tochicin, 10.5 kDa, effective against over 20 B. thuringiensis members (Paik et al. 1997). B. thuringiensis B439 produces two antibiotic peptides, thuricin 439A and 439B (Ahem et al. 2003), both <3 kDa, differing by 100 Da. Torkar and Matijasic (2003) report several bacteriocins, 1-8 kDa, from B. cereus milk isolates and B. cereus ATCC 14579 produces a BLIS (bacteriocin like inhibitory substance) with a molecular weight of 3.4 kDa (Risoen et al. 2004). B. cereus BC7 produces cerein 7, 3.94 kDa (Oscariz et al. 1999) and B. cereus strain 8A, from the soils of Brazil, produces cerein 8A (Bizani and Brandelli 2002).

Bacteriocins such as those described above may be tested for plant growth and/or disease resistance promoting activity as described herein.

The polypeptides of the invention may also be obtained from bacterial species that are known to have plant growth promoting activity or to produce compounds that promote plant growth, but that are not necessarily known to produce bacteriocins. These include, for example, plant growth promoting rhizobacteria (PGPR). PGPR increase plant growth and include bacteria in the soil near plant roots, on the surface of plant root systems, in spaces between root cells or inside specialized cells of root nodules (Kloepper et al., 1978).

Some PGPRs are known to produce bacteriocins, and bacteriocin production by PGPR members is illustrated by Pseudomonas ssp. (Parret and De Mot 2002) and bacteriocins denoted as “rhizobiocins” from rhizobia (Schwinghamer 1975). Rhizobium leguminosarium bv. viciae strain 306 produces the bacteriocin, pR1e306c, with a type I secretion system required for export (Venter et al. 2000). Wilson et al. (1998) found a R. leguminosarum isolate that produces a virulent bacteriocin lethal to 68% of soil isolate strains. The bacteriocin may have facilitated its persistence in the soil (Wilson et al. 1998).

PGPR can be classified as extracellular PGPR (ePGPR) or intracellular PGPR (iPGPR) based on their degree of association with plants (Gray and Smith, 2005). iPGPR are the nodulating rhizobia housed within the cells of anatomically sophisticated nodules and provide reduced nitrogen to plants. ePGPR are those that reside in the soil, on the surface of plants or in the extracellular spaces in plant root tissue. ePGPR increase plant growth through a broad range of mechanisms, for instance by producing phytohormones (Bastian et al., 1998; Jameson, 2000) or metal chelating siderophores (Carson et al., 2000) and by suppressing disease through antibiosis (Maurhofer et al., 1992). Both ePGPR and iPGPR may be used in the practice of the invention. Illustrative examples of ePGPR include Pseudomonas, Lactobacillus and Bacillus species, while illustrative examples of iPGPR include the rhizobia (species in the genera, for example, Rhizobium, Sinorhizobium, and Bradyrhizobium species such as Bradyrhizobium japonicum), or species of Frankia.

The foregoing is not limiting and proteins from other sources (for example fungi, protists or cyanobacteria) may be tested for bactericidal and/or bacteristatic activity as well as plant growth and/or disease resistance-promoting activity.

In an embodiment, the polypeptide is obtained from or obtainable from Bacillus (e.g. B. thuringiensis or B. cereus), Pseudomonas, Rhizobium, or Bradyrhizobium.

In an embodiment, the polypeptide is a class IID bacteriocin.

In one embodiment the polypeptide is a polypeptide that is obtained from or obtainable from Bacillus thuringiensis, especially Bacillus thuringiensis strain NEB 17, originally isolated from soybean root nodules (Bai et al. 2003), and which was deposited at the International Depositary Authority of Canada (IDAC) on Mar. 27, 2003 under Accession No. 270303-02. Thuricin17, discussed below, and Bacthuricin F4 are two novel bacteriocins having plant growth and/or disease resistance promoting activity isolated by the inventors from B. thuringiens is strain NEB17 and their uses are contemplated herein.

In one embodiment, the polypeptide is a bacteriocin (designated BF4) which is obtainable from or obtained from B. thuringiensis strain BUPM4. In another embodiment, the polypeptide is a bacteriocin (designated C85) which is obtainable from or obtained from B. cereus strain UW85. BF4 (strain BUPM4) and C85 (strain UW85) bear strong likenesses to T17 (strain NEB17). Each of these bacteriocins does not kill the strains that produce the other two, indicating the same mechanism of action (and the same mechanism for protecting against T17, BF4 and C85). T17, F4 and C85 have HPLC elution times that, while not identical, are very similar. While the total amino acid composition indicates differences between T17 and BF4, the first 17 amino acids from the amino end are the same. UW85 has been deposited in the American Type Culture Collection under accession number ATCC 53522. BUPM4 is in the collection of the Medical Faculty of Sfax, in Tunisia.

As used herein, by “obtainable” it is meant that the polypeptide is equivalent (i.e. has the same amino acid sequence) to one expressed by the mentioned bacterial strain but is not limited to the polypeptide only when produced by that strain. For instance, the polypeptide could be produced recombinantly in a host cell or organism or synthesized chemically.

In one embodiment the polypeptide may possess one or more of the following properties:

(a) the polypeptide may maintain bactericidal and/or bacteristatic activity after exposure to a temperature of 100° C. for at least 15 minutes;

(b) the polypeptide may maintain bactericidal and/or bacteristatic activity after treatment with α-amylase or catalase, but exhibit loss of activity after treatment with proteinase K or protease; and

(c) the polypeptide may have molecular weight in the range of about 3100 to 3200 Da.

In one embodiment, the polypeptide is a novel polypeptide denoted thuricin 17 (T17) identified by the inventors. T17 comprises the partial amino acid sequence WTCWSCLVCAACSVELL (SEQ ID NO: 1). In one embodiment such a polypeptide is obtained from or obtainable from Bacillus thuringiensis strain NEB 17 (IDAC 270303-02).

In one embodiment, the polypeptide is a polypeptide that retains at least some of the bacteriocin and plant growth and/or disease resistance promoting activity of T17 but differs in sequence from T17 by one or more amino acid insertions, deletions, or substitutions, particularly conservative amino acid substitutions. As used herein, the terms “conservative amino acid substitutions” refers to the substitution of one amino acid for another at a given location in the polypeptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

Accordingly, such a polypeptide may possess at least one activity of a bacteriocin and plant growth promoting activity and comprise a region, preferably a region of 17 consecutive amino acids, that possesses at least 70, 80, 90, 95, 96, 97, 98, or 99% identity to SEQ ID NO: 1 over the entire length of SEQ ID NO: 1, when optimally aligned. The term “identity” refers to sequence similarity between two polypeptide or polynucleotide molecules. Identity can be determined by comparing each position in the aligned sequences. A degree of identity between amino acid or nucleic acid sequences is a function of the number of identical or matching amino acids or nucleic acids at positions shared by the sequences, for example, over a specified region. Optimal alignment of sequences for comparisons of identity may be conducted using a variety of algorithms, as are known in the art, including the ClustalW program, available at http://clustalw.genome.adjp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm (e.g. BLASTn and BLASTp), described in Altschul et al., 1990, J. Mol. Biol. 215:403-10 (using the published default settings). Software for performing BLAST analysis is available through the National Center for Biotechnology Information (through the internet at http://www.ncbi.nlm.nih.gov/).

Naturally occurring variant sequences may be more likely to retain bacteriocin and plant growth and/or disease resistance promoting activities, such as homologs produced by closely related bacterial species.

The polypeptides are preferably in purified form. By “purified” is meant that the polypeptide is substantially separated or isolated from the components such as other polypeptides, proteins, or lipids, carbohydrates, etc. that accompany the polypeptide in its natural environment. Thus, for example, a polypeptide that is chemically synthesised or produced by recombinant technology will generally be substantially free from its naturally associated components and be considered to be purified. Typically, the purified polypeptide will constitute at least 60%, 70%, 75%, 80%, 90%, 95%, 98% or 99% by weight, of the total material in a sample (i.e. a sample of the purified polypeptide will contain less than 40%, 30%, 20%, 10%, 5%, 2% or 1% by weight of components such as other polypeptides, proteins, lipids, carbohydrates, etc. that accompany the polypeptide in its natural environment). A substantially purified polypeptide can be obtained, for example, by extraction from a natural source, by expression of a recombinant polynucleotide encoding the polypeptide compound or by chemical synthesis. Purity can be measured using any appropriate method such as column chromatography, gel electrophoresis, HPLC, etc.

In one embodiment, polypeptides may be obtained from bacterial species that express the polypeptides. For instance, the bacterial strain may be cultured under conditions sufficient for expression of the polypeptide and the polypeptide recovered from the culture medium. The polypeptide may be purified by e.g. by chromatography (e.g. high-performance liquid chromatography), gel electrophoresis, filtration, dialysis, precipitation, centrifugation, etc. or combinations thereof. In a preferred embodiment, the polypeptide is purified by solid phase extraction, e.g. using a C18 solid phase extraction column such as a PREPSEP C18 column (Fisher Scientific, Pittsburgh Pa., USA).

The polypeptides may be expressed recombinantly, by culturing a host cell transformed or transfected with nucleic acid encoding the polypeptide. The host cell may be a prokaryotic host cell, for example a bacterial cell, or a eukaryotic cell, such as a yeast, plant, or animal cell.

Alternatively, the polypeptides may be synthesized chemically via known procedures.

In another embodiment, the invention provides polynucleotides encoding the polypeptides of the invention. The term “polynucleotide” refers to a polymeric form of nucleotides of any length and may also be referred to in the art as a “nucleic acid” or “nucleic acid molecule”. The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the invention include full-length genes and cDNA molecules as well as a combination of fragments thereof.

The polynucleotides of the invention are preferably “isolated” polynucleotides by which it is meant that they are not presents in their naturally occurring form associated with the 5′ and/or 3′ sequences with which they are normally found. The polynucleotides are separated from at least one or both of the 5′ or 3′ sequences with which they are normally associated. For example, a nucleic acid molecule of the invention, inserted into a vector or linked to a foreign promoter, is in “isolated” form.

The invention also provides vectors, such as plasmid vectors, viral vectors, expression vectors, etc. comprising the polynucleotides of the invention, as well as host cells transformed or transfected with polynucleotides of the invention. The host cells may be host cells as described above.

Fragments of the isolated nucleic molecules of the invention, having lengths of at least about 10, 12, 14, 16, 18, 20, 25, 30, 35, 40, 50, 60, 70, 80, 90 or 100 nucleotides are encompassed by the invention and are useful as e.g. probes in hybridization reactions to identify polypeptides related to thuricin 17 that have bacteriocin and plant growth and/or disease resistance promoting activity or as PCR primers for amplifying such sequences.

Polypeptides may be applied either before, during or after planting and may be applied to, for example, plant leaves, stems, roots, or seeds. As used herein and in the claims, the term “plant” includes without limitation whole plants, plant parts, organs, leaves, stems and roots. For greater certainty, plant seeds are discussed separately in the claims as it is envisaged that the plant growth and/or disease resistance promoting compositions may be applied to the seeds well e.g. in advance of planting. The polypeptide may additionally or alternatively be applied to the growing environment of the plant or seed rather than directly to the plant or seed. By “growing environment” is meant the area sufficiently proximal to the plant or seed (such as to the soil adjacent to the plant or seed) that the polypeptide can effect a growth- or disease resistance-promoting effect on the plant. If the polypeptide is applied to the soil, it may be applied before, during or after planting.

The polypeptide may be applied by any suitable means, either in solid (e.g. as a free-flowing powder) or liquid form (such as in an aqueous carrier). Leaf spray and root irrigation are two preferred techniques. The polypeptide may also be applied to various portions of the plant or seed in slow-release formulations, such as beads or gels. The skilled person can readily determine suitable application regimes for the polypeptide. In one embodiment, the polypeptide is applied in an aqueous carrier at a concentration of about 10⁻⁹, 10⁻¹⁰ or 10⁻¹¹ M, equivalent to a total of 15.8, 1.58 and 0.158 ng pot⁻¹ (where each pot contained ten plants), respectively.

In practicing the methods of the invention, the polypeptides may be used alone or in the form of a plant growth and/or disease resistance promoting composition. Such compositions may contain diluents, adjuvants, excipients, carriers, etc. suitable for inclusion in a plant growth and/or disease resistance promoting compositions as are known in the art. The compositions may be in, for example, solid (such as powdered) or liquid form. The plant growth and/or disease resistance promoting composition may be provided in the form of a kit containing the composition and e.g. instructions for use of the composition for promoting plant growth.

The composition may take the form of plant seeds pre-treated with the plant growth and/or disease resistance promoting composition.

Plants are able to synthesize a broad range of secondary metabolites capable of improving their resistance to pathogen attack. In many cases these are only synthesized when the plants are exposed to compounds that indicate the presence of the pathogen (Somssich et al., 1986)—elicitors such as oligosaccharides.

The major molecular events of plant-pathogen interactions can be divided into three steps: i) generation and recognition of signal compounds, ii) inter- and intracellular signal conversion and transduction, and iii) activation of signal-specific responses in target cells (Ebel and Cosio, 1994). Various elements of the multi-component plant defense mechanism induced by elicitors include the hypersensitive reaction (HR) (Artat et al., 1994), the production of activated oxygen species (oxidative burst) (Apostol et al., 1989), the modification of plant cell walls by deposition of callose (Conrath et al., 1989), and the synthesis and accumulation of antimicrobial phytoalexins (Dixon et al., 1983). In addition to these localized defenses, systemic acquired resistance (SAR), which increases the plant's resistance to subsequent pathogen attack, is activated in many plants; it can also be induced by specific elicitor compounds (Somssich et al., 1986).

Elicitor molecules produced by microorganisms are extremely diverse in nature. Four major classes of elicitor-active oligosaccharides have been identified as oligoglucan, oligochitin, oligochitosan from fungi and oligogalacturonide of plants (Cote and Hahn, 1994). Chitin is an elicitor molecule produced by fungal cell walls; it is a polysaccharide and is composed of β-1-4-linked N-acetylglucosamine units. Glucans, which have the ability to stimulate the production of phytoalexins, newly synthesized antimicrobial compounds of low molecular weight, were initially detected in culture filtrates of the oomycete Phytophthora sojae, a pathogen of soybean (Ayers et al., 1976). Glucans similar to those active as elicitors in soybean occur as extracellular polysaccharides in the symbiotic partner of soybean, Bradyrhizobium japonicum (Rolin et al., 1992). Cyclic β-1,3-1,6-glucans of the microsymbiont of soybean, Bradyrhizobium japonicum USDA 110 have been shown to have elicitor activity (Miller et al., 1990).

Accordingly, elicitors of plant pathogen defense mechanisms may be used in conjunction with the methods and compositions of the invention. Such elicitors may be applied to plants, seeds, or the growing environment of the plant together with or separately from the polypeptide possessing plant growth and/or disease resistance promoting activity. Plant growth and/or disease resistance promoting compositions of the invention may contain such elicitors, or be packaged together with the elicitor. Preferred elicitors include oligosaccharides, such as oligoglucans, oligochitins, oligochitosans, (preferably from fungi) and oligogalacturonides.

Plants planted, germinated or grown in the presence of the plant growth and/or disease resistance promoting polypeptides of the invention may exhibit an increase in plant growth, such as an increase in one or more of nodulation, nitrogen fixation, height, increased seedling emergence, leaf area, seed germination, leaf greenness, photosynthesis, or shoot, root, or total dry weight, relative to a plant that has not been treated with the plant growth and/or disease resistance promoting composition.

Similarly plants planted, germinated or grown in the presence of the plant growth and/or disease resistance promoting polypeptides of the invention may exhibit one or more characteristics of improved disease resistance, such as, for example, reduced or inhibited pathogen infestation, increased activity of a lignification-related enzyme such as PAL or TAL or an antioxidative enzyme such as POD, CAT or SOD. Increases of enzyme activity of more than 10, 20, 30, 40, 50, 60, or 70% may be obtained by the methods of the invention. Increases in concentration of total phenolics of more than 1, 5, 10, 15 or 20% may be obtained by the methods of the invention.

The compositions of the invention may be used and the methods of the invention practiced wherever plants are grown, such as in greenhouse, field, or laboratory conditions. The compositions may be used with plants that are grown at temperatures above 30° C., at which temperatures nitrogen fixing rhizobacteria are generally most active, or also at low temperatures, such as at an average daily root zone temperature below 28, 26, 24, 22, 20, 18, 16, 14, 12, or 10° C.

The methods of the invention are not limited to use with any particular plant or plant-type. Exemplary plants with which the methods of the invention may be practiced include, without limitation: legumes, such as soybeans, peanuts, pulses (e.g. peas and lentils), beans, forage crops (e.g. alfalfa and clover), plants of lesser agricultural importance (e.g lupines, sainfoin, trefoil, and even some small tree species); tomato plants; corn; horticultural tree species (e.g. peach, apple, plum, pear, mango), forestry tree species (e.g. spruce, pine, fir, maple, oak, poplar).

The polypeptides of the invention may also be used as bacteriostatic and/or bactericidal agents in any application in which it would be desirable or advantageous to prevent or inhibit growth of bacteria.

For example, the polypeptides of the invention may be used to treat or prevent bacterial infection in a subject, such as a mammalian subject, especially a human subject. In this embodiment, the polypeptide may be formulated as a pharmaceutical composition comprising a polypeptide of the invention together with one or more pharmaceutically acceptable carriers, diluents or excipients. Such compositions may include additional bactericidal and/or bacteriostatic agents as are known in the art. Pharmaceutical compositions may be formulated for administration, for example, topically, intravenously, orally, rectally, parenterally, etc. Suitable dosages and routes of administration can be determined by the skilled person.

The polypeptides of the invention may also be employed to inhibit or prevent growth of bacteria in other applications, such as on a surface, in a liquid, in a nutrient medium, in a food product, etc., and the polypeptide may be formulated into a bactericidal and/or bacteristatic composition comprising the polypeptide together with one or more suitable carriers, excipients and diluents, and optionally one or more additional bactericidal and/or bacteristatic agents.

The invention is further exemplified by the following non-limiting examples.

### EXAMPLES

### Example 1

### a) Bacterial Strains and Culture Preparations

Bacillus thuringiensis NEB17 (BtNEB17) was cultured in King's Medium B consisting of proteose peptone #3 (20 g L⁻¹), K₂HPO₄ (0.66 g L⁻¹), MgSO₄ (0.09 g L⁻¹) and glycerol (0.06 mL L⁻¹) (Atlas 1995). The initial broth inoculum was taken from plated material and grown in 250 mL flasks, containing 50 mL of medium. The bacterium was cultured at 28±2° C. on an orbital shaker (Model 5430 Table Top Orbital Shaker, Form a Scientific Inc., Mariolta, Ohio, USA) for 48 h, rotating at 150 rev min⁻¹. A 5 mL sample of subculture was added to 2 L of broth and cultures were grown in 4 L flasks under the same conditions as for the initial culture. Bacterial populations were determined spectrophotometrically using an Ultrospec 4050 Pro UV/Visible Spectrophotometer LKB (Cambridge, England) at 600 nm (Dashti et al. 1997) 96 h after culture preparation. A cell free supernatant (CFS), containing the BtNEB 17 compound, was prepared by centrifuging the bacterial culture at 13,000 g for 10 min on a Sorvall Biofuge Pico (Mandel Scientific). The supernatant was collected and the bacterial compound was detected via analytical-HPLC on a Vydac C: 18 reversed-phase column (0.46×25 cm; 5 μM) (Vydac, Calif., USA; catalogue # 218TP54). The HPLC was fitted with Waters 1525 Binary HPLC pump and a Waters 2487 Dual λ Absorbance detector set at 214 nm. All other bacterial strains, sources and culture media are given in Table 1 below.

### b) Extraction and Partial Purification of the Bacterial Peptide

For partial purification of the bacterial peptide, BtNEB17 cells were cultured as described above. Two liters of bacterial culture was phase partitioned against 0.8 L butanol for 12 h. The upper butanol layer was collected and evaporated using the rotary evaporator (Yamota RE500, Yamato, USA) at 50° C. under vacuum. After evaporation, the resulting light brown viscose extract was resuspended in 25 mL of 18% acetonitrile (AcN:H₂O, v/v). Prior to HPLC analysis, samples were centrifuged on a Sorvall Biofuge Pico (Mandel Scientific) at 13,000 g for 13 min, and the supernatant collected for chromatography. HPLC analysis (Waters, Mass., USA) was conducted on a Vydac C: 18 reversed-phase column as described above.

Conditions of the fractionation chromatography were as follows: 45 minutes at 18% acetonitrile, 45 to 110 minutes of gradient elution with 18 to 60.4% of acetonitrile, 110 to 115 minutes at 60.7 to 100% of acetonitrile and 115 to 120 minutes at 100 to 18% of acetonitrile. The HPLC elutions were collected at 1 minute intervals (Bai et al. 2002b). Preparative HPLC samples were separated into 120 minute fractions and were analyzed for peaks with retention times between 80 and 82 minutes, as this is when the peptide elutes. The peptide elutes in approximately 60% acetonitrile, and is denoted as partially purified bacterial peptide (PPBP). As a control, purified culture media, without btNEB17, was subjected to the same purification procedures.

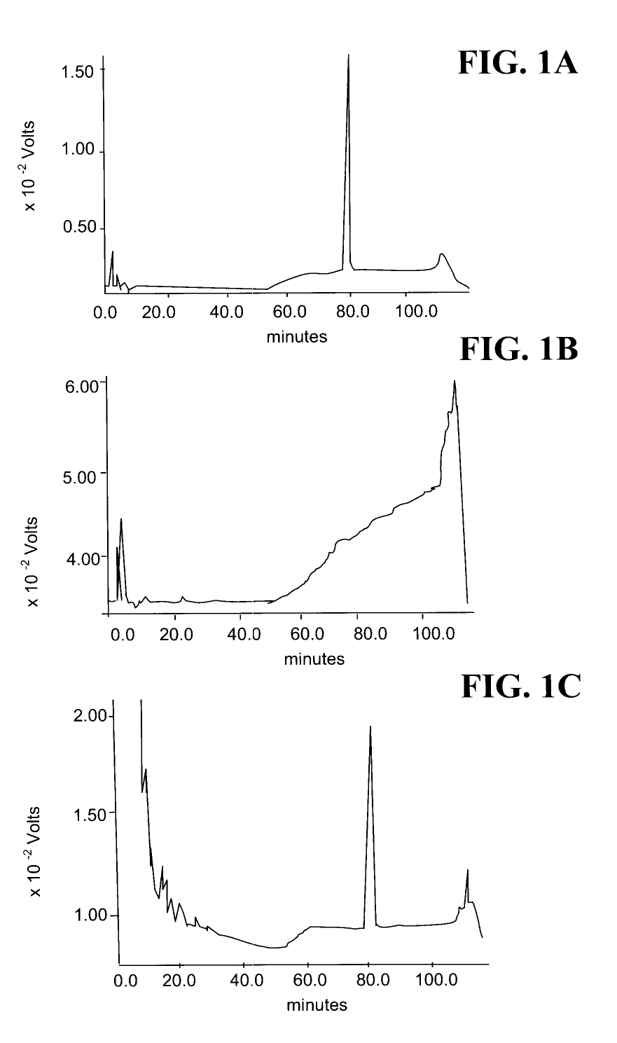

As shown in FIG. 1, PPBP and CFS shows a distinctive peak when analyzed via HPLC and in both cases the peak retention times were 80-82 min (FIG. 1A and FIG. 1C, respectively). In purified culture media without Bacillus thuringiensis NEB17 this peak is absent (FIG. 1B).

### c) Initial Characterization of the Bacterial Peptide

The BtNEB17 compound was initially assessed for protein content via the Bradford assay (Bradford 1976). Aliquots of 2 mL of PPBP with retention times of 80-82 min were lypholized at −60° C., under vacuum pressure. This was conducted using a Savant Modulyo Freeze-dryer fitted with a Savant Model VPOF oil pump and Savant Model VPL200 air pump. Two hundred μL of ddH₂O were added to samples and the Bradford assay was performed with samples being read for absorbance at 595 nm.

### d) Inhibition Range and Activity Assessment

Antimicrobial activity of the BtNEB17 peptide was assessed via agar disk diffusion assay (Kimura et al. 1998) on all indicator strains listed in Table 1 below. In the present example, to assess the range of antimicrobial activity, a host of Bacillus members and non-Bacillus members were tested for their inhibition by the BtNEB17 peptide (Table 1). The peptide was inhibitory to other Bacillus strains, including 16/19 B. thuringiensis strains, 4/4 B. cereus strains, 2/2 B. megatarium strains, 2/3 B. licheniformis strains and 1/2 B. pumilus strains (Table 1). Other inhibited species/strains were Brevibacillus brevis, Geobacillus stearothermophilus, 2/2 Paenibacillus polymyxa and Escherichia coli MM294 (pBS42). Bacillus strains not inhibited included 0/3 B. subtilis plus the plant growth promoting strains listed (Table 1).

TABLE 1
Antimicrobial spectrum of the bacterial peptide using the disk diffusion assay,
where 15 μL of CFS were spotted on to sterilized 6 mm disks. The assay was
done twice with two separate biological replicates, in duplicate (n = 4).

| | | Zone of |
|---|---|---|
| | | Inhibition in |
| Indicator Species^{a} | Source* | Diameters (mm) |
| Bacillus thuringiensis NEB17^{†} | SLC¹ | — |
| Bradyrhizobium japonicum 532C^{‡} | USDA² | — |
| Bradyrhizobium japonicum USDA 3^{‡} | USDA² | — |
| Bradyrhizobium japonicum USDA 110^{‡} | USDA² | — |
| Escherichia coli MM294(pBS42) £ | BGSC³ | 7.5 |
| Escherichia coli JM83(pMK3) £ | BGSC³ | — |
| Pseudomonas putida NRLL-B-14688^{†} | ARSCC⁴ | — |
| Ralstonia spp. H16 ATCC 17699^{†} | ATCC⁵ | — |
| Serratia proteomaculans 1-102^{†} | SLC¹ | — |
| Serratia proteomaculans 2-68R^{†} | SLC¹ | — |
| Stenotrophomoas meiltophilia Alfa-nod^{†} | KU⁶ | — |
| Staphylococcus epidermidis ATCC 12228^{†} | ATCC⁵ | — |
| B. thuringiensis subsp. thuringiensis HD2^{†} | BGSC³ | 10.5 |
| B. thuringiensis subsp. kurstaki HD1^{†} | BGSC³ | — |
| B. thuringiensis subsp. sotto 4-1^{†} | BGSC³ | 10 |
| B. thuringiensis subsp. galleriae HD29^{†} | BGSC³ | 10 |
| B. thuringiensis subsp. canadensis HD224^{†} | BGSC³ | 8 |
| B. thuringiensis subsp. entomocidus HD10^{†} | BGSC³ | 13 |
| B. thuringiensis subsp. entomocidus HD9^{†} | BGSC³ | — |
| B. thuringiensis subsp. subtoxicus HD109^{†} | BGSC³ | — |
| B. thuringiensis subsp. morrisoni HD12^{†} | BGSC³ | 8 |
| B. thuringiensis subsp. darmstadiensis HD146 (103)^{†} | BGSC³ | 9 |
| B. thuringiensis subsp. pakistani HD395^{†} | BGSC³ | 15 |
| B. thuringiensis subsp. indiana HD521^{†} | BGSC³ | 9.5 |
| B. thuringiensis subsp. tochigiensis HD868 (117-72)^{†} | BGSC³ | 7 |
| B. thuringiensis subsp. cameroun 273B^{†} | BGSC³ | 11 |
| B. thuringiensis serovar. xiaguangiensis 3397^{†} | BGSC³ | 10 |
| B. thuringiensis serovar. asturiensis EA 34594^{†} | BGSC³ | 7 |
| B. thuringiensis serovar. rongseni Scg04-02^{†} | BGSC³ | 12 |
| B. thuringiensis subsp. thuringiensis Bt1627^{†} | BGSC³ | 19 |
| B. thuringiensis subsp. alesti HD4^{†} | BGSC³ | 13.5 |
| B. cereus T-HT^{†} | BGSC³ | 13 |
| B. cereus T-HW3^{†} | BGSC³ | 7 |
| B. cereus 6A3 StrepR^{†} | BGSC³ | 18 |
| B. cereus ATCC 14579^{†} | BGSC³ | 14 |
| B. licheniformis Alfa-Rhiz^{†} | USDA² | — |
| B. licheniformis 9945A^{†} | BGSC³ | 9 |
| B. licheniformis 749^{†} | BGSC³ | 7.5 |
| B. megaterium ATCC 19213^{†} | BGSC³ | 18 |
| B. megaterium QM B1551^{†} | BGSC³ | 14 |
| B. pumilus ATCC 7061^{†} | BGSC³ | 9 |
| B. pumilus Biosubtyl^{†} | BGSC³ | — |
| B. sphaericus 1593^{†} | BGSC³ | — |
| B. subtilis NEB 5^{†} | SLC¹ | — |
| B. subtilis NEB 4^{†} | SLC¹ | — |
| B. subtilis subsp. subtilis 168^{†} | BGSC³ | — |
| Bacillus KTCC B1^{†} | KU⁶ | 12 |
| Bacillus KTCC B2^{†} | KU⁶ | 13 |
| Aneurinibacillus migulanus NRS-1137T^{†} | BGSC³ | — |
| Brevibacillus brevis ATCC 8246^{¥} | BGSC³ | 17 |
| Geobacillus stearothermophilus 10^{¥} | BGSC³ | 11 |
| Paenibacillus polymyxa ATCC 842^{¥} | BGSC³ | 10.5 |
| Paenibacillus dendritiformis C168^{¥} | BGSC³ | 9 |

KTCC: Korean Type Culture Collection,
NEB: Non-Bradyrhizobium endophytic bacterium.
^{†}Strains cultured in King's Medium (Atlas 1995),
^{‡}Strains cultured in Yeast Extract Mannitol (Vincent 1970),
£ Strains cultured on MacConkey Agar (Difco),
^{¥}Strains cultured on Tryptose Blood agar (Oxoid).
Source*: SLC¹: Dr. Smith Laboratory Collection, Department of Plant Science, McGill University, Montreal, Quebec, Canada; USDA²: United States Department of Agriculture, USA; BGSC³: Bacillus Genetic Stock Center, University of Ohio, Department of Biochemistry, Cleveland, Ohio, USA; ARSCC⁴: Agricultural Research Service Culture Collection, Peoria, Illinois, USA; ATCC⁵: American Type Culture Collection; KU⁶: Kuwait University, Department of Biology, Kuwait, Kuwait.

Indicator strains were cultured and tested for purity prior to running the assay and were then streaked onto agar plates. Due to the large volumes of material required, two replicates of the CFS were tested, instead of the PPBP. 15 μL of sample was spotted onto sterilized disks (6 mm) and allowed to dry. Petri dishes were maintained for at least 48 h at 27° C. after which the zone of inhibition was measured (mm). Media types and components for the varying indicator strains consisted of King's Medium; YEM (Vincent 1970) consisting of: mannitol (10.0 g L⁻¹), K₂HPO₄ (0.5 g L⁻¹), MgSO₄ (0.1954 g mL⁻¹), NaCl (0.1 g L⁻¹), yeast extract (0.4 g L⁻¹) and agar (15%); MacConkey's (Microbiology, Germany) agar prepared according to suppliers instructions, with 5 μg mL⁻¹ of chloramphenicol for Escherichia coli MM294(pBS42) and 50 μg mL⁻¹ of ampicillin for E. coli JM83(pMK3); tryptose Blood Agar, prepared according to manufacturers instructions (Difco, USA): tryptose blood agar base (10 g L⁻¹), NaCl (4.8 g L⁻¹), agar (12 g L⁻¹) and sterile defribinated sheeps' blood (72 mL L⁻¹).

The activity of the BtNEB17 peptide was quantified by using a series of two fold dilutions (modified from Mayr-Harting et al. 1972) and was conducted on separate replicates. Briefly, 15 μL of two-fold dilution factors were spotted onto sterilized disks (6 mm) and allowed to dry; duplicates were conducted for each sample. The specific activity of samples was calculated as the reciprocal of the highest dilution that gave a clearly visible inhibition zone. This was expressed in activity units (AU) and determined using the indicator strain B. cereus ATCC 14579. By weighing lypholized peptide an estimate of peptide concentration (μg L⁻¹) was determined and compared with the AU.

### e) Mode of Action Assessment

The mode of action was assessed following the methods of Ahem et al. (2003). Briefly, subculture strains of B. thuringiensis ssp. thuringiensis Bt1627 and B. cereus ATCC 45679 were grown in King's medium (Atlas 1995) to an O.D. ₆₀₀ₙₘ of 0.35-0.40. At this time, cultures were diluted, with sterilized medium, to an O.D. ₆₀₀ₙₘ of 0.27-0.30. Cultures were then divided into 10 mL aliquots and placed into 30 mL flasks. Volumes of 0, 100, 300 and 600 μL of PPBP (0.066 μg μl⁻¹) were added to the cultures. B. thuringiensis NEB17 was cultured in the same manner and exposed to the same treatments as a negative control. Cell density O.D. ₆₀₀ₙₘ was then read using an Ultrospec™ 4050 Pro UV/Visible Spectrophotometer LKB (Cambridge, England). Results were confirmed by the number of viable colony forming units (CFU) log mL⁻¹. Briefly, subsamples of cell cultures were taken each hour and diluted in 0.9% NaCl solution, 50 μL of diluted bacterial culture was inoculated onto agar plates, and viable cell count determined. Values are expressed on a log scale. The entire experiment was also repeated with CFS (0.071 μg μL⁻¹).

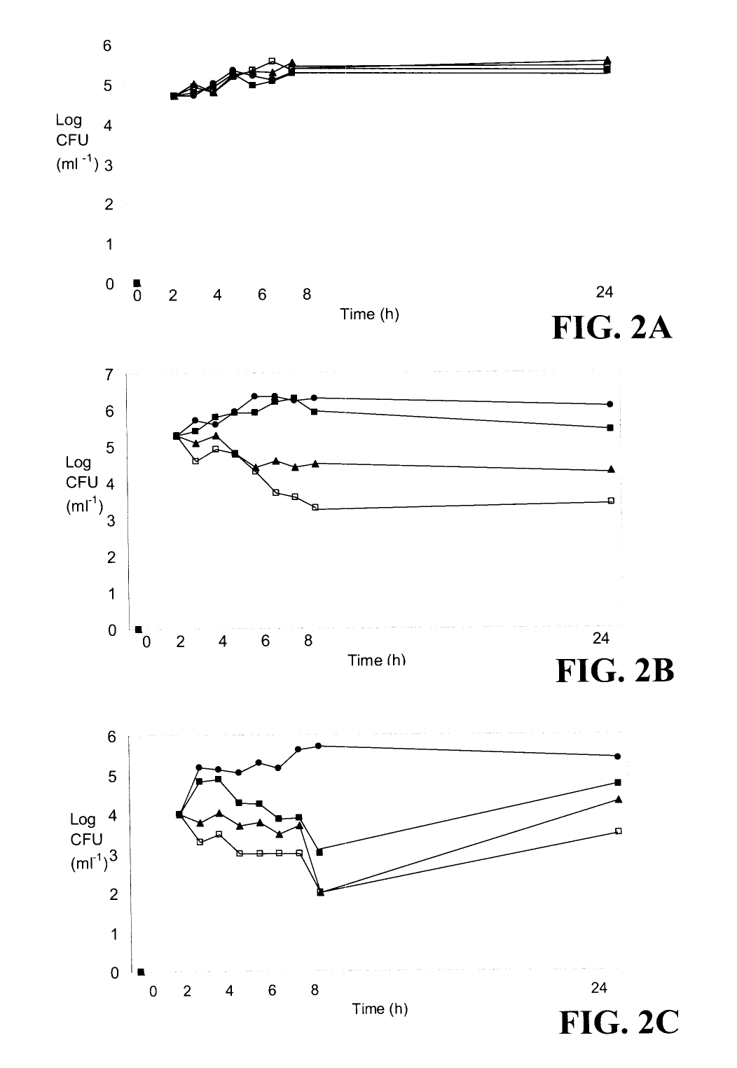

An assessment of the mode of action confirmed that the bacterial peptide is both bactericidal and bacteristatic (FIGS. 2A-C). From the onset of exposure, cell density of both B. thuringiensis spp. thuringiensis Bt 1627 (FIG. 2C) and B. cereus ATCC 14579 (FIG. 2B) decreased continually and cell lysis eventually occurred. The static effect was observed on B. cereus ATCC 14579 (FIG. 2B), while B. thuringiensis spp. thuringiensis Bt 1627 (FIG. 2C) was able to recover showing a later increase in growth. B. thuringiensis NEB17 (FIG. 2A) served as a negative control and there was no effect on the producer strain. Results were consistent between experiments using the CFS and the PPBP.

### f) SDS-PAGE and Direct Inhibition

Samples of PPBP and CFS were run on a SDS-PAGE gel and compared against a protein standard (1.4-26.6 kDa, Bio-Rad catalogue #161-0326). Purified medium (passed through HPLC purification and collected at 80-82 min), centrifuged medium, and 6× loading dye were controls. An aliquot of either PPBP or CFS was diluted in a 1:1 (vol/vol) SDS/sample buffer and denatured by heating for 5 min at 100° C. For direct detection of BtNEB17 peptide, activity, PPBP, CFS, purified medium and centrifuged medium (all without added sample buffer and loading dye) were run on the same gel. Samples were run on a 22% polyacrylamide gel using a tris-glycine running buffer at pH 8.3, 200 V, and 50 mA gel⁻¹ (changed after 15 min to 30 mA gel⁻¹). After electrophoresis, the gel was fixed for 30 min in 25% isopropanol and 10% acetic acid and sliced vertically. The first half was stained with Coomassie Blue (100 mL acetic acid, 900 mL ddH₂O:methanol (1:1), 2.5 g Coomassie blue G-250) for 1 h. It was then destained with two washings of 100 mL acetic acid and 900 mL ddH₂O:methanol (1:1), then left to destain overnight. The second half of the gel was soaked in several changes of distilled water for overnight and overlaid with soft agar in a Petri dish. Direct detection of the BtNEB17 peptide was determined using the indicator strain, Bacillus thuringiensis ssp. thuringiensis Bt 1267. Briefly, 300 μL of culture containing the indicator strain was inoculated onto the plate. The Petri plate was maintained at 27° C. for at least 48 h.

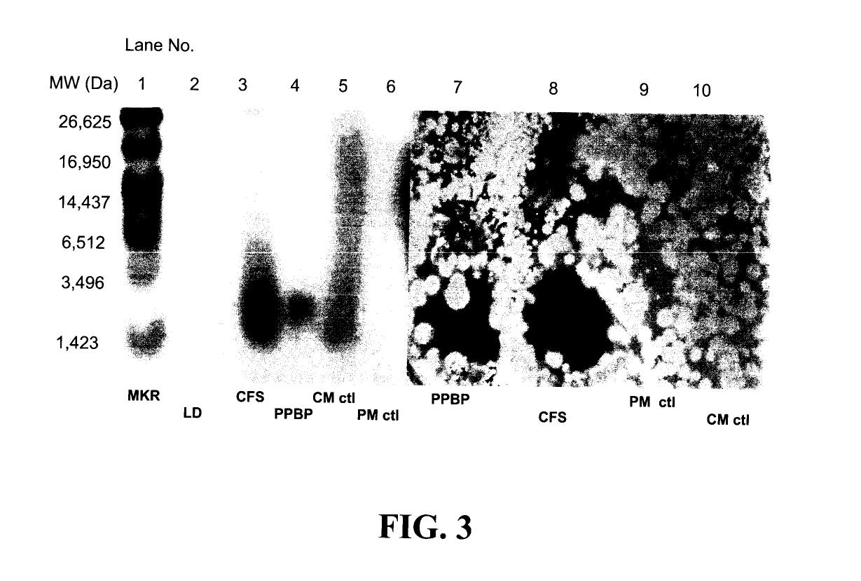

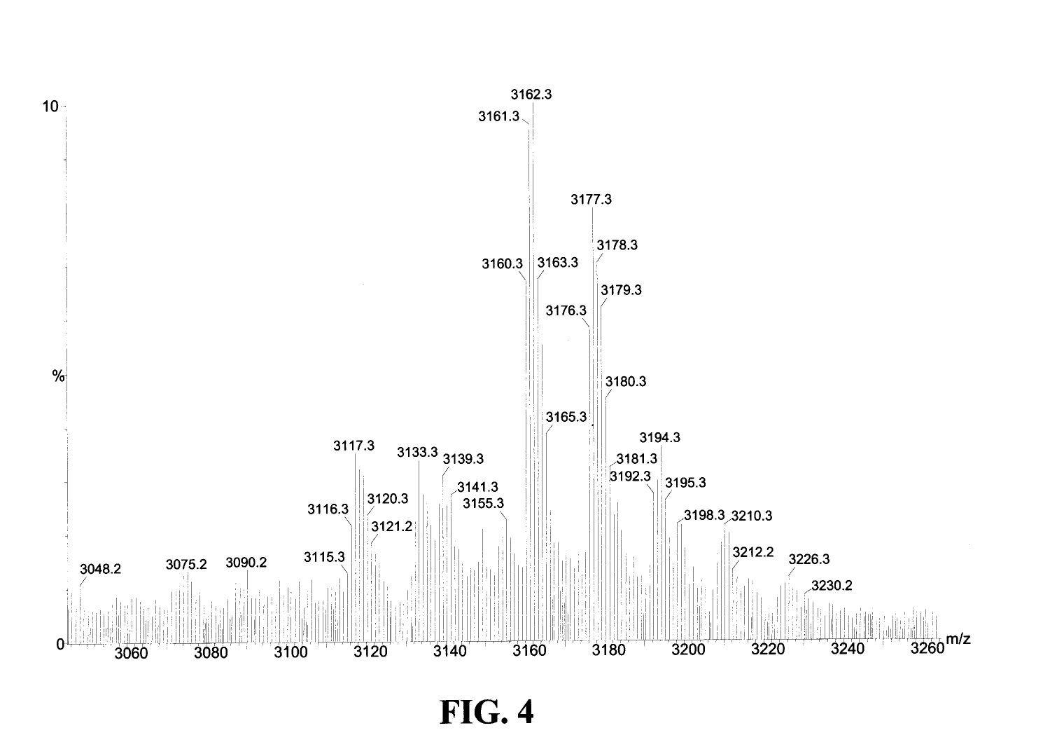

SDS-PAGE indicated that the peptide present in the PPBP and CFS weighed 2500-3000 Da (FIG. 3, lanes 3 and 4). Results show it is also responsible for directly inhibiting bacterial growth. Due to the high percentage of acrylamide in the gel, it took many attempts to grow the indicator strain and colonies appear as an uneven lawn. Despite this, the inhibitory effects of the peptide were observed and it is inferred that the BtNEB17 peptide is responsible for direct inhibition of bacterial growth. SDS-PAGE provided an estimate of the peptide's molecular weight and MALDI mass spectrometry data confirmed these results. A strong mass peak from MALDI analysis is observed at 3162.3 Da (See FIG. 4 below). Additional testing, using FAB mass spectrometry, yielded similar results (data not shown).

### g) Enzyme Degradation/Heat and pH Stability Assays

Separate samples of the PPBP and CFS samples were digested with Proteinase K (from Tritirachium album, Sigma No. P-2308), Protease (from Streptomyces griseus, Sigma No. P-6911), α-amylase (from barley malt VIII-A, Sigma No. A-2771) and catalase (from Corynebacterium glutamicum, Sigma No. 02071). Upon enzymatic digestion the PPBP was then tested for antimicrobial activity. Working buffer solutions and pH levels were as follows: proteinase K-100 mol L⁻¹ Tris-HCL, pH 7.5 at 20° C.; protease-0.04 mol L⁻¹ potassium phosphate (equal volumes of monobasic/dibasic), pH 7.5 at 20° C.; α-amylase-0.02 mol L⁻¹ sodium phosphate (monobasic) plus 0.06 mol L⁻¹ NaCl, pH 6.9 at 20° C.; catalase-0.05 mol L⁻¹ potassium phosphate (monobasic), pH 7.0 at 20° C. For proteinase K, protease and α-amylase enzymes were added to final concentrations of either 1 mg mL⁻¹ or 2 mg mL⁻¹. Catalase was added at either 40,000 U mL⁻¹ or 60,000 U mL⁻¹. Samples were incubated for 120 min at 37° C., then heated at 100° C. for 2 min for enzyme inactivation. Controls were as follows: PPBP plus the corresponding enzyme buffer, CFS plus the corresponding enzyme buffer, enzymes in corresponding buffer, purified medium and centrifuged medium.

Heat stability assays were conducted on the CFS and PPBP. A 250 μL sample of material was heated for 30 min at the following temperatures: 40, 60, 75, 100, and 121° C. 250 μL were also exposed to −20° C. for 30 days, 4° C. for 30 days and 22° C. for 24 h. To assess an effective pH range, 10 mL of CFS were subjected to a range of pH from 1.00-13.75 (modified from Oscariz et al. 1999). As large volumes of material were needed for this assay the CFS were used to determine pH stability, instead of the PPBP. Centrifuged medium adjusted to the same pH was a control. The pH levels were determined using an Accumet Dual Channel pH/Ion Conductivity Meter model AR50 (Fisher Scientific, Montreal). Inhibitory activity was conducted at 21° C. and assessed on the indictor strain Bacillus thuringiensis ssp thuringiensis Bt 1627 and/or B. cereus ATCC 14579 (Table 2).

The biological activity of the PPBP and the CFS disappeared completely when exposed to 2 mg mL⁻¹ of protease and almost completely when treated with proteinase K (data not shown). Exposure to 1 mg mL⁻¹ of proteinase K and protease resulted in partial loss of activity (data not shown). However, no loss of activity was seen when treated with either 1 or 2 mg mL⁻¹ of α-amylase. Catalase when added at 40 000 U ml⁻¹ or 60 000 U ml⁻¹ had no effect on the activity of the PPBP and the CFS. To ensure that any degradation and/or denaturation resulting in loss of bioactivity was not due to heat treatment used in the enzyme assay, controls were run, wherein PPBP and CFS were exposed to 37° C. for 2 h and 37° C. for 2 h plus 100° C. for 2 min (Table 2). No loss in bioactivity was found when the PPBP and CFS were exposed to these conditions, ensuring that the loss of activity during the proteinase K and protease digestion assays were actually due to the enzyme degradation.

Both the CFS and PPBP were stable over a wide heat range, and were resistant to degradation when exposed to 100° C. for 15 min (Table 2). They were also stable when kept at −20° C. for 30 days, at 4° C. for 30 days and resistant to lypholization. The pH stability was between 1.00 and 9.25. At higher pH levels, the biological activity disappeared, and the peptide was not effective as a bacteriocin. Results from the activity assessment show the CFS had 32 AU (0.071 μg μL⁻¹). This was repeated on a new culture of bacteria and another CFS was generated and results were similar, 64 AU (0.059 μg μL⁻¹).

TABLE 2
Characterization of the PPBP in response
to varying temperature and pH levels.

| pH | Duration | | % Activity* |
|---|---|---|---|
| 1.00 | 3 | h | 50 |
| 1.25 | 3 | h | 91 |
| 1.50 | 3 | h | 100 |
| 1.75 | 3 | h | 100 |
| 2.00 | 3 | h | 100 |
| 4.00 | 3 | h | 100 |
| 6.00 | 3 | h | 100 |
| 8.00 | 3 | h | 100 |
| 9.00 | 3 | h | 73 |
| 9.25 | 3 | h | 50 |
| 9.50 | 3 | h | 0 |
| Temp. (C.°) | Duration | | % Activity** |
| −20 | 30 | days | 100 |
| 4 | 30 | days | 100 |
| 22 | 24 | h | 100 |
| 37 | 2 | h | 100 |
| 45 | 30 | min | 100 |
| 60 | 30 | min | 100 |
| 80 | 30 | min | 94 |
| 100 | 2 | min | 100 |
| 37 + 100 | 2 h + 2 min† | | 100 |
| 100 | 315 | min | 95 |
| 121 | 5 | min | 0 |

*Activity was determined via disk diffusion assay on one separate biological replicate of the CFS and on one separate biological replicate of PPBP
**†Samples were incubated at 37° C. for 2 h, followed by incubation at 100° C. for 2 min. Tested on the indicator strain, B. thuringiensis ssp. thuringiensis Bt 1627 and/or B. cereus ATCC 14579.

### h) Mass Spectroscopy

Prior to analysis, PPBP samples were lypholized as described above. Analysis was conducted at the Genome Quebec and McGill University Innovation Center, using an Ultima MALDI (Matrix Assisted Laser Desorption Ionization)—QTOF (Quadrapole Time of Flight) mass spectrophotometer (Waters Corp., Milford, Mass.). For comparison of methods, additional mass spectrometry analysis was also conducted on a fast-atom bombardment mass spectrometry (FAB-MS) in positive mode in a JEOL-SX/SX102A mass spectrometer (JOEL Inc., Toyko, Japan). Both types of mass spectrometry analysis were conducted on material isolated from different BtNEB17 cultures, grown at different times.

As discussed above, SDS-PAGE provided an estimate of the bacterial peptide's molecular weight. MALDI mass spectrometry data confirmed these results. A strong mass peak from MALDI analysis is observed at 3162.3 Da (FIG. 4). Additional testing, using FAB mass spectrometry, yielded similar results (data not shown).

### Example 2

### Peptide Sequence and Production of T17 by NEB17

### a) Bacterial Strains and T17 Isolation

Bacillus thuringiensis NEB17 (NEB17) was cultured in King's Medium B: Proteose peptone #3 (20 g L⁻¹), K₂HPO₄ (0.66 g L⁻¹), MgSO₄ (0.09 g L⁻¹) and glycerol (0.06 mL L⁻¹) (Atlas 1995). The bacterial cultures were grown in 4 L flasks containing 2 L of liquid media for at least 72 h at 28±2° C. on an orbital shaker (Model 5430 Table Top Orbital Shaker, Form a Scientific Inc., Mariolta, Ohio, USA). Cultures were grown until an O.D. ₆₀₀ₙₘ of at least 1.4 (or approximately 5.5 log CFU (colony forming units) cells per mL) as determined using spectrophotometry Ultrospec™ 4050 Pro UV/Visible Spectrophotometer LKB (Cambridge, England).

T17 partial purification was conducted by phase partitioning 2 L of bacterial with 0.8 L butanol for 12 h. The aqueous layer was removed and the organic layer concentrated at 50° C. under vacuum by rotary evaporation (Yamota RE500, Yamato, USA). The remaining material was then resuspended in 25 mL of 18% acetonitrile (AcN:H₂O, v/v). Prior to purification, all material was stored in a sterilized, sealed vial at 4° C. Purified media alone, without added bacteria, was subjected to the same extraction protocol, and this material acted as a control.

For HPLC analysis samples were centrifuged on a Sorvall Biofuge Pico™ (Mandel Scientific) at 13,000 g for 13 minutes, and the supernatent collected for chromatography. The HPLC (Waters, Mass., USA) had a Vydac C18 reversed-phase column and the gradients of acetonitrile:water during the fractionation were as follows: 45 min. at 18% acetonitrile, 45 to 110 min. 18 to 60.4% acetonitrile; 110 to 115 min, 60.7 to 100% acetonitrile, then finally, 115 to 120 min at 100 to 18% acetonitrile. The material was collected at 1 min intervals (Bai et al. 2002b), as it has been previously shown that T17 elutes between 80 and 85 min. (Gray et al. 2006a).

### b) Protein Sequencing

Protein sequencing was conducted at McGill University and at the Virginia Bioinformatics Institute. Edman degradation for N-terminal sequencing was conducted on a Procise Applied Biosystems 492 gas-phase/pulsed-liquid automated sequencer. PTH (phenylthiohydantoin) derivatized amino acid residues were then analyzed on a C: 18 HPLC column. The amino acid sequence was then assigned using the software program Model 610A. Sequencing was conducted one time each on two separate biological replicates of material from NEB17. However, there was a sudden stop in the sequence after the 18^{th} cycle during each run.

Attempts to digest the peptide with carboxypepsidase Y were unsuccessful, along with digestion attempts with trypsin (added at a concentration of 2 μg/200 μL of peptide), which gave sufficient amounts of activity of chromotrypsin. However, a successful combination of carboxypepsidase Y and W allowed the generation of a C-terminal ladder, in which two additional amino acids were cleaved and the possibility of a third, though the signal was riot as strong. The sequence then stopped at that point.

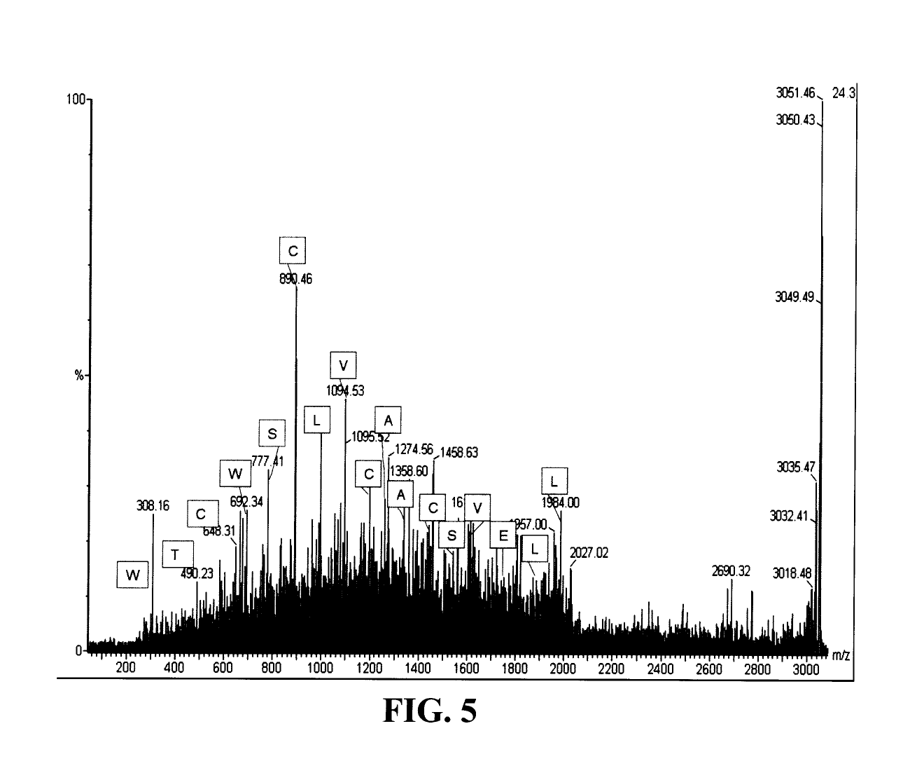

Sequence determination of the thuricin 17 peptide was conducted via a combination of Edman Degradation based N-terminal sequencing and tandem mass spectrometry. The data showed the N-terminal sequence as follows: WTCWSCLVCAACSVELL (SEQ ID NO: 1). During the Edman Degradation the analysis stopped after the 18^{th} cycle and did this consistently in both repetitions. The positions of cysteines within the sequence were not determined by N-terminal sequencing since this amino acid is degraded during the Edman sequencing reaction. Instead they were determined by ms/ms fragment analysis of the parent ion, of mass 3051 Da (FIG. 5). In determining the sequence, the 3061 Da ion was used. The molecular weight of the ion for sequencing was slightly less than the initially determined molecular weight of 3162 Da (Gray et al. 2006a). It was difficult to fragment the ion for sequencing and in fragmenting the intact peptide, partial amino acid residues were lost at the site of a putative site of post-translational modification (PTM). Nonetheless, we are still able to obtain partial sequence data which does coincide with amino acid analysis.

A signal drop-off (difficulty in sequencing past a specific amino acid residue) has also been reported for other bacteriocins. Ahern et al. (2003) found a signal drop off at the 20^{th} cycle when trying to sequence both thuricin 439A and 439B. They proposed the presence of cysteine in the sequence that was obtained, as no signal could be determined at some points during the Edman degradation. Furthermore, the sequence information for Plantaricin S, a bacteriocin produced from Lactobacillus plantarum LPCO10 requiring the complementary action of two peptides, was obtained up to amino acid residues 26 and 24; a PTM may have prevented further sequencing (Jimenez-Diaz et al. 1995).

The peptide was then treated with carboxypepsidase Y and trypsin to generate peptide ladders for mass spectrometry based C-terminal sequencing. However, the peptide was resistant to further digestion (data not shown). Again, this is not uncommon. The activity of T7, from B. thuringiensis BMG1.7, was inhibited by proteinase K, but not with trypsin (Cherif et al. 2001). A BLIS from B. cereus ATCC 14579 is resistant to trypsin, RNAse and lysozyme, but not to proteinase K and pronase E (Risoen et al. 2004). Coagulin (Hyronimus et al. 1998) is resistant to degradation by trypsin. Exposure of thuricin 17 to carboxypepsidase Y and W yielded sufficient fragments for C-terminus analysis. A C-terminus sequence of CAS—C-terminus was then determined.

### c) Amino Acid Analysis

Amino acid analysis was performed at the University of Virginia Health Center. Briefly, the peptide was hydrolysed in 6NHCL vapor for 24 h at 110° C., to free the amino acids. During this assay, glycoprcoteins, containing a carbohydrate (CHO) moiety, will often turn black, as carbon is oxidized. This can indicate the presence of a CHO on the protein. Derivatization occurred; this yields phenylthiocarbamyl (PTC) amino acids that are analyzed via HPLC, where the instrument is fitted with a C:18 reverse phase column.

Amino acid analysis results coincided, for the most part, with the sequence data. The amino acid analysis detected the presence of 1-Asx, 1-Glx, 3-Ser, 1-Gly, 4-His, 2-Thr, 7-Ala, 3-Val and 4-Leu, which yields an estimated molecular weight of 3242+1H₂O, for a total of 3260 Da. Interestingly these provide an overestimate of the molecular weight by 100 Da. This may be explained in that the configuration of amino acids in the presumed PTM(s) is not known. This suggests a PTM of 100 Da that was undetected during the initial mass spectrometry analysis. Furthermore, in digesting the peptide some amino acids could be counted more than once. It was thought that perhaps the remaining weight was due to a CHO (carbohydrate) moiety, which was the PTM blocking the Edman degradation. However, the presence of a CHO moiety results in a black color upon acid hydrolysis and this did not occur, making the presence of CHO unlikely. Thus, it was confirmed that T17 does not contain a CHO component and its molecular weight is probably composed entirely of amino acids, perhaps with a 100 Da PTM.

BLAST searches on the sequence show some homology to other bacterial peptides such as Sinorhizobium meliloti 1021 complete chromosome; segment 6/12 Identities=8/13 (61%), Positives=10/13 (76%), Xanthomonas campestris pv. campestris str. ATCC 33913, Identities=7:13 (53%), Positives=11/13 (84%), Rhodobacter sphaeroides, Identities=10/16 (62%), Positives=10/16 (62%), Neisseria meningitidis MC58, Identities=8/12 (66%), Positives=11/12 (91%). However, no exact match was found via BLAST searches, and in comparison with existing sequence information on currently published bacteriocins, confirming that T17 is a novel compound.

### d) Thuricin 17 Production

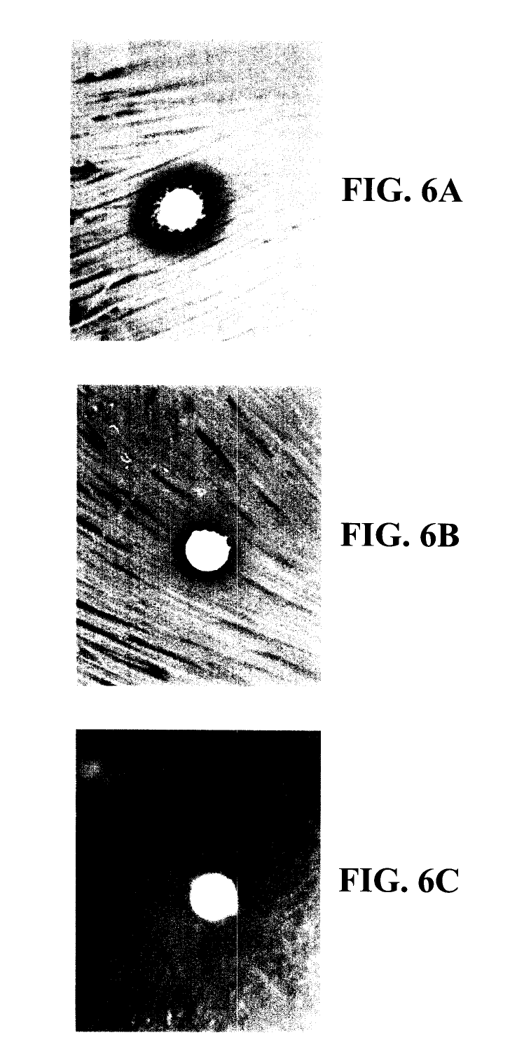

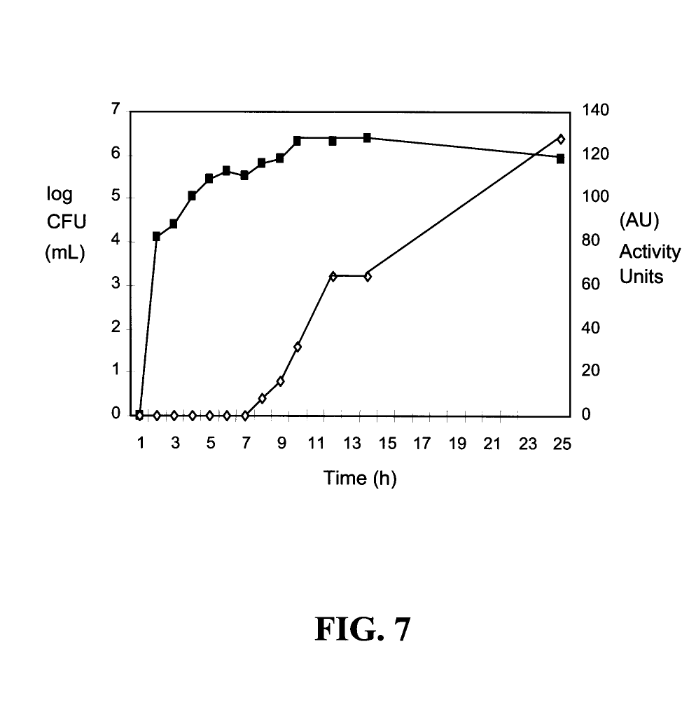

Production of the material by B. thuringiensis NEB17 was determined by preparing subcultures of cells taken from Petri plates and culturing for at least 12 h. One mL of this material was then added to 250 mL of King's medium. Subsamples were taken every hour and the O.D. ₆₀₀ₙₘ (Optical Density) and log CFU (Colony Forming Units) mLU were determined (FIG. 7). The O.D. was determined spectrophotometrically with an Ultrospec™ 4300 Pro UV/Visible Spectrophotometer. The CFU was determined by diluting subsamples, taken each hour, in 0.9% NaCl solution. Fifty μL of diluted bacterial culture was then inoculated onto agar plates, and viable cell count determined. The activity of T17 was quantified as specific activity units (AU) using the indicator strain, B. thuringiensis ssp. thuringiensis Bt 1627. This was done by preparing a CFS (Cell Free Supernatant), extracting material every hour, preparing a series of two fold dilutions. For detection of inhibition, the disk diffusion assay was used; 15 μL of diluted T17 was spotted onto sterilized filter paper disks (6 mm diameter). Production of T17 begins at the mid-exponential growth phase and continues well into the stationary phase (FIG. 7), which coincides with the results for thuricin, B. thuringiensis HD2 (Favret and Yousten 1989) and thuricin 7, B. thuringiensis BMG1.7 (Cherif et al 2001). Initial traces of T17 were found at an O.D. ₆₀₀ₙₘ of 1.3 (FIG. 6B). The concentration then continued to increase as the stationary phase continued. The AU was calculated as the reciprocal of the highest dilution that gave a visible inhibition zone. It has been previously shown that bacteriocin production occurs in the mid-logarithmic growth phase; these include entomocin 9, B. thuringiensis ssp. entomocidus HD9 (Cherif et al. 2003) and tochicin, B. thuringiensis HD868 (Paik et al. 1997).

### Example 3

To determine whether or not thuricin 17 plays a role in plant growth enhancement by NEB17, this study determined the effect of thuricin 17 on soybean photosynthesis and growth under controlled environment conditions.

### a) Isolation of Thuricin 17

Bacillus thuringiensis NEB17 was cultured in King's Medium B (Altas, 1995). A stock culture of bacteria was grown in 250 mL flasks, containing 50 mL of broth. Bacteria were cultured at 28±2° C. on an orbital shaker (Model 5430 Table Top Orbital Shaker, Form a Scientific Inc., USA) for 32 h, rotating at 150 rpm. Culture populations were determined at 600 nm using an Ultrospec 4300 Pro UV/Visible Spectrophotometer (Biochem Ltd., England), then adjusted with broth to a 1% inoculation ratio (final volume) in 4.0 L flasks containing 1.0 L of the broth culture medium. The resulting subculture was grown for 48 h. Subcultures were separated by differential centrifugation (Sorvall RC 5C Plus, Mandel Scientific Co., USA) for 20 min at 2,800×g and 4° C. Butanol, to 60% the final volume, was added and the mixture was shaken at 4° C. overnight. The mixture was then allowed to stand for 2 h, while the two phases separated, after which the upper butanol layer was collected. The butanol was removed by rotary evaporation (Yamota RE500, Yamato, USA) at 50° C. under vacuum. The resulting viscose extract was resuspended in 18% acetonitrile (AcN:H₂O, v/v) and further purified through HPLC (Waters 510 system, Waters, USA). The HPLC was equipped a C₁₈ reverse-phase column (Vydac218TP54, 300 nm, 5 μm, 4.6×250 mm), model 441 absorbance detector at 214 nm and column temperature at 20° C. The elution was performed as follows: 0-45 min with isocratic 18% AcN and 45-110 min with a gradient from 18 to 60.7% AcN. HPLC eluates were collected as 110 fractions, 1 min of elution time per fraction, and maintained at 4° C. until use. Culture medium, without bacteria, was put through the same extraction and purification procedure, and the resulting material was used as a negative control.

### Bacteriocin Properties of Thuricin 17

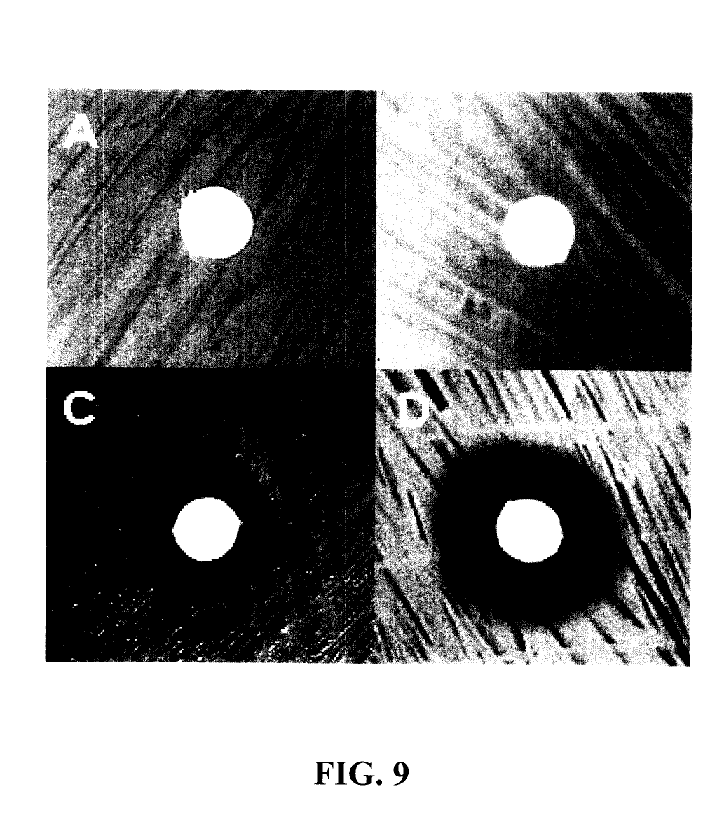

To ensure that the material being tested was the bacteriocin thuricin 17, bacterial strains were tested for their inhibition by thuricin 17. This was done via the disk diffusion assay with strains cultured on King's Medium B (Atlas, 1995), solidified with 15% agar (FIG. 9). Petri plates containing medium were inoculated with strains susceptible to thuricin 17 (Gray et al, 2006a) and 15 μL of thuricin 17 (10⁻⁷ M) was spotted onto sterilized disks (6 mm diameter) (FIGS. 9C and D). Plates were then maintained at 28° C. for at least 48 h. Controls were the producer strain, B. thuringiensis NEB17 and partially purified media (FIGS. 9A and B).

### b) Plant Bioanalysis of Thuricin 17

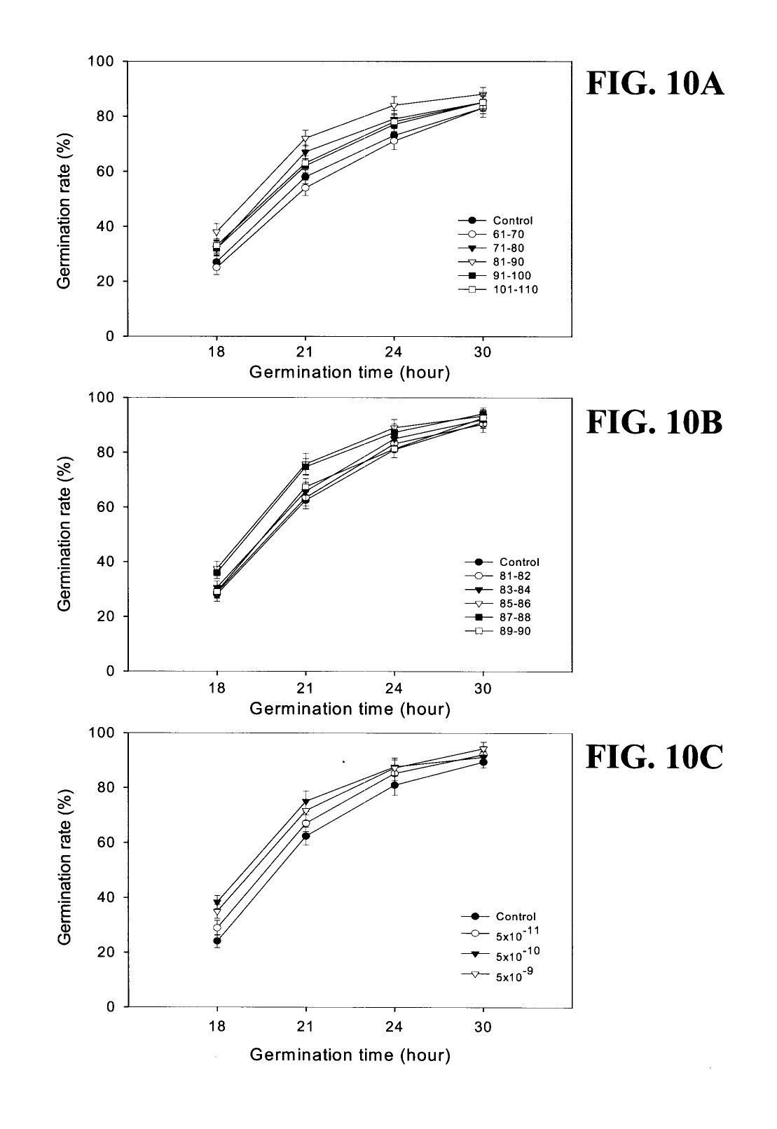

The 110 collected fractionations were initially assayed to assess their plant biological activity. In the first step, fractions 61 to 110 were aggregated into 5 groups (61-70, 71-80, 81-90, 91-100 and 101-110 minute fractions; FIG. 10A), pooled and tested for their ability to enhance seed germination of soybean cultivar OAC Bayfield. The active fractions selected in the first step (81-90 minute fractions) were further divided into five groups (81-82, 83-84, 85-86, 87-88 and 89-90 minute fractions; FIG. 10B) and retested. Soybean seeds were surface-sterilized in 2% sodium hypochlorite for 3 min and then rinsed 5 times with distilled water (Bhuvaneswari et al., 1980). Ten soybean seeds were placed on two layers of sterilized filter paper wetted with 7 mL of treatment solution, in Petri dishes. Treatment application marked the beginning of the assay. Petri dishes were maintained in an incubator (Conviron E15 Growth Chamber, Controlled Environments Ltd., Winnipeg, Canada) at 25±1° C. and 70-80% humidity. Germination was determined to have occurred when the root tip had clearly penetrated the seed coat. The number of germinated seeds was recorded periodically for 30 h and germination was expressed as a percentage (%) of the total number of seeds in the dish.

Once the bioactivity of thuricin 17 had been established, the concentration causing the greatest increase in germination was determined. Thuricin 17 solutions were prepared by lyophilizing purified material at −60° C., under vacuum pressure using a Savant Modulyo Freeze-dryer fitted with a Savant Model VPOF oil pump and Savant Model VPL200 air pump. The dried fraction was then resuspended in sterilized, distilled water. As the molecular weight of this compound has been determined (Gray et al., 2006a) concentrations of thuricin 17 are given in Molar and the applied concentrations were 0, 5×10⁻¹¹, 5×10⁻¹⁰ and 5×10⁻⁹ M, (water control, T17-1, T17-2 and T17-3, respectively) (FIG. 10C). The germination assay was conducted as described above and the entire experiment conducted twice.

### c) Greenhouse Experiments

Based on results from the germination assay, thuricin 17 was investigated for its ability to enhance soybean nodulation, photosynthesis and growth under greenhouse conditions. Soybean seeds of OAC Oxford (an early maturing cultivar) and Korada (a late maturing cultivar) were surface-sterilized in 2% sodium hypochlorite for 3 min, and rinsed 5 times with distilled water (Bhuvaneswari et al., 1980). These two cultivars were selected as they have been widely grown in eastern Canada. Seeds were placed in sterilized vermiculite to germinate. Seven days after seeding, at the VE (emergent) stage (Fehr et al., 1971), seedlings were transplanted into 13 cm pots, each containing 100 g of sterilized dry vermiculite, at a rate of 1 seedling per pot. Four days after transplanting, healthy seedlings were inoculated with B. japonicum 532C (BJ 532C).

BJ 532C was cultured in yeast extract mannitol culture medium (YEM) (Vincent, 1970). Broth was inoculated with slant material and cultured on an orbital shaker at 150 rpm for 7 days at 28° C. A subculture was prepared by inoculating new broth medium with the initial culture such that the added inoculant material constituted 1% of the volume of the subculture. After 5 days the subculture was centrifuged at 2,800×g for 20 min at 4° C. Cell density was estimated by spectrophotometry at 620 nm (Bhuvaneswari et al., 1980) and the broth was diluted with sterilized tap water to A₆₂₀=0.08 (approximately 10⁸ cells mL⁻¹), and the inoculation dose was 10⁸ cells per seedling (Zhang and Smith, 1994).

Thuricin 17 was applied to soybean plants by either leaf spray or root irrigation. In both types of application thuricin 17 was applied at 0, 5×10⁻¹¹ (T17-1), 5×10⁻¹⁰ (T17-2) and 5×10⁻⁹ M (T17-3). Treatments were applied three times to each plant, when soybean plants were at the V1, V2 and V3 stages (Fehr et al., 1971). For leaf sprays, Tween 20 (0.01%) was added into treatment solutions and also the control. The top surfaces of the pots were covered with vinyl plastic to ensure the treatment solutions did not drip onto the soil. Treatment solutions were sprayed, with an atomizer, onto leaves until wet. For the largest plants this was equivalent to 1 mL per plant, with smaller amounts for smaller (earlier stage of development) plants. For soil irrigation, treatment solutions, including the control, did not contain Tween 20. Treatment solution, 1 mL, was diluted with distilled water to become 20 ml, and poured on the rooting medium surface at the base of the plant stem. Plants were grown for 40 days following the initial application of treatment solutions.

During the growth period, plants were watered daily with half strength nitrogen-free Hoagland's solutions (Hoagland and Amon, 1950), in which the Ca(NO₃)₂ and KNO₃ were replaced with 0.5 mM CaCl₂, 0.5 mM K₂HPO₄, and 0.5 mM KH₂PO₄ to provide nitrogen free nutrient solution. The greenhouse temperature was 25±2° C., relative humidity was 75% and a 16 h photoperiod was created by supplemental lighting from high-pressure sodium lamps. At each harvest, data were collected on plant height, leaf greenness (SPAD-502, Minolta, Japan), leaf area (Delta-T Devices, Cambridge, UK), nodule number and nodule dry weight, shoot and root dry weight (Zhang and Smith, 1995). Shoot, root and nodule tissues were air-dried at 60° C. for 5 days for determination of dry weight. Nitrogen content and photosynthesis were measured using an NC 2500 Elemental Analyzer (CE Instrument Inc., Italy) and Li-Cor 6400 (Li-Cor Inc, USA), respectively.

### d) Statistical Analysis

The pot experiment was structured as a randomized complete block design (RCBD) with four replications. Data were analyzed via analysis of variance (ANOVA) using CoStat software (CoStat Software, Monterey, USA). Since there was no interaction between cultivar and application method, cultivar and concentration, or cultivar, application method and concentration, but there was an application method by concentration interaction, data are presented as application method by thuricin 17 concentration interaction means. Means comparisons were conducted using an ANOVA protected the least significant difference (LSD) (P<0.05) test.

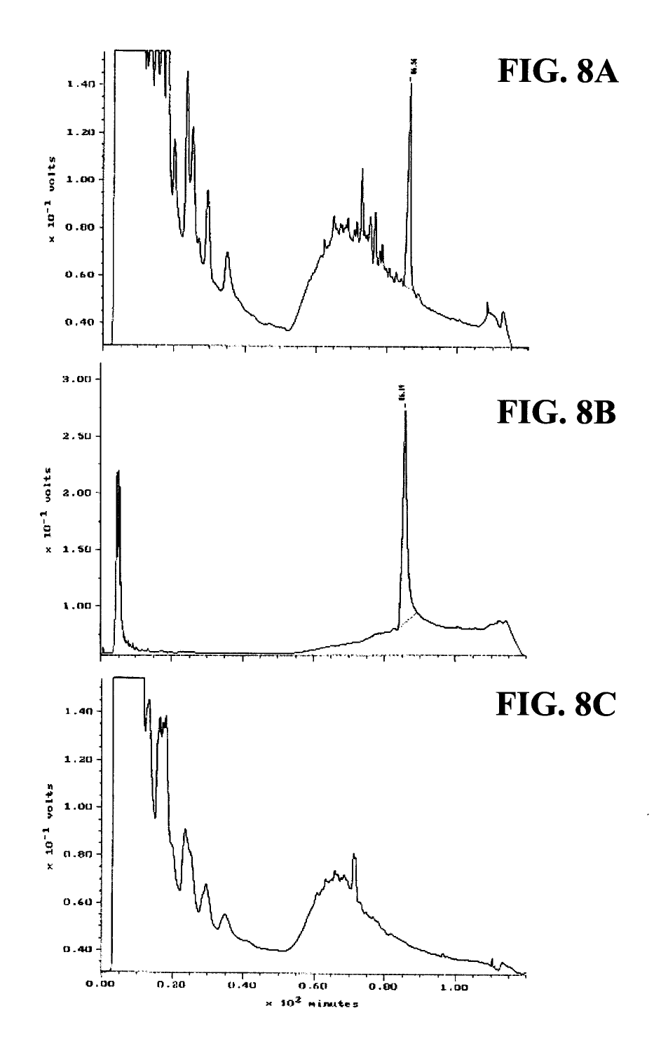

Initial purification of thuricin 17 began with analysis of the crude extract of bacterial medium in which the PGPR strain Bacillus thuringiensis NEB17 had been grown. The crude extract showed a peak that corresponded to thuricin 17 (FIG. 8A). HPLC partial purification of thuricin 17 showed a distinctive peak on the chromatogram at approximately 85 minutes (FIG. 8B). This peak was not present for control medium that had not grown B. thuringiensis NEB17 (FIG. 8C). The distinctive thuricin 17 peak facilitates isolation and recognition during the purification process. The thuricin 17 isolated and used in this experiment was bactericidal to the closely related strains B. cereus ATCC 14579, (FIG. 9C), and Brevibacillus brevis ATCC 8246, (FIG. 9D), confirming its bacteriocin nature. However, thuricin 17 did not inhibit the growth of B. thuringiensis NEB17 (FIG. 9A), the thuricin producer, or B. japonicum 532C.

The germination assay showed that material in fractions collected at 85-88 min caused the greatest stimulation of germination (FIGS. 8A and 8B). This HPLC retention time corresponds to that of thuricin 17. The concentration of thuricin 17 that caused the greatest stimulation of germination, relative to the medium extract control, was 10⁻¹⁰ M (FIG. 10).

In greenhouse studies there w ere no interactions between cultivar and application method, cultivar and concentration, and among cultivar, application method and concentration. However, there was an interaction between application method and concentration of thuricin 17, hence means are presented for this interaction. When applied as a leaf spray thuricin 17, treatment T17-2 increased leaf photosynthetic rates about 6% over the control (from 13.75 to 14.55 μmol cm⁻² s⁻¹) (Table 3). Leaf greenness (SPAD reading) was similarly affected and the average value for T17-2 was 29.2, as compared with the control at 27.3 (Table 3). Increases in leaf area were also observed for all three treatments, with T17-2 causing the greatest increase. T17-2 increased plant dry weight by 15% from 1.137 for the control to 1.304 g plant⁻¹ in the T17-2 treatment (Table 3).

When applied to roots, all three thuricin 17 treatments increased photosynthetic rates, as compared with the control and T17-2 had the greatest effect. Leaf greenness (SPAD reading) was increased by all thuricin 17 treatments, with T17-1 having the greatest effect. Leaf area (cm² plant⁻¹) was also increased by thuricin 17 (Table 3). The greatest increase was due to treatment T17-1, being 173.0, followed by T17-2, 169.9, as compared with the control at 155.9 (Table 3). Plant dry weight increased from 1.147 (control) to 1.278 for T17-1 and 1.250 g for T17-2. Application of thuricin 17 did not affect plant height (Table 3).

TABLE 3
Effects of thuricin 17 on soybean (cultivars OAC Oxford and
Korada) photosynthesis, leaf greenness, leaf area, plant
height and plant dry weight at harvesting time. The application
method (leaf spray and root irrigation) and concentration
interaction means are shown.

| | Photo- | | | | | | |
|---|---|---|---|---|---|---|---|
| | synthetic | Leaf | Leaf area | Plant | Dry weight | | |
| | rate | color | cm² | height | Shoot | Root | Total |
| Treatment | μmol cm⁻² s⁻¹ | (SPAD) | plant⁻¹ | cm | g plant⁻¹ | | |
| Leaf spray | | | | | | | |
| Control^{a} | 13.75 c^{c} | 27.3 d | 154.3 d | 13.9 | 0.817 c | 0.320 | 1.137 c |
| T17-1^{b} | 13.86 c | 28.1 c | 163.6 bc | 14.1 | 0.860 bc | 0.327 | 1.187 bc |
| T17-2 | 14.55 a | 29.2 a | 175.2 a | 14.5 | 0.942 a | 0.362 | 1.304 a |
| T17-3 | 14.41 ab | 28.7 abc | 171.1 ab | 14.3 | 0.928 a | 0.357 | 1.285 a |
| Root | | | | | | | |
| irrigation | | | | | | | |
| Control | 13.63 c | 27.3 d | 155.9 cd | 14.1 | 0.830 c | 0.317 | 1.147 c |
| T17-1 | 14.43 ab | 29.3 a | 173.0 a | 14.3 | 0.920 a | 0.358 | 1.278 a |
| T17-2 | 14.44 a | 29.0 ab | 169.9 ab | 14.2 | 0.893 ab | 0.357 | 1.250 ab |
| T17-3 | 14.03 bc | 28.4 bc | 156.6 cd | 14.1 | 0.838 c | 0.350 | 1.188 bc |

^{a}Means were based on leaf spray treatments containing the surfactant Tween 20, while treatments for root irrigation did not.
^{b}T17-1, T17-2 and T17-3 represent thuricin 17 concentrations of 5 × 10⁻¹¹, 5 × 10⁻¹⁰ and 5 × 10⁻⁹ M, respectively.
^{c}Means within the same column and factor followed by the same letter are not different (P ≦ 0.05) by an ANOVA-protected LSD test.
When letters are absent, ANOVA indicated no difference among means (n = 4).

Direct application of thuricin 17 to leaf tissue (Table 4) increased nodule number (P<0.05). T17-2 increased nodule number to 103.6 nodules plant⁻¹, an 18% over the control plants. However, application of thuricin 17 to leaves did not affect nodule dry weight. Nitrogen concentration (mg g⁻¹ dry weight) in shoot tissue was increased by thuricin 17 treatments T17-2 and T17-3. However, thuricin 17 did not affect root N concentrations. The pattern of effects was similar for total fixed N (mg plant⁻¹), in that there were effects of leaf spray with 45.58 and 45.51 mg of fixed N per plant for T17-2 and T17-3, respectively, versus 35.16 mg fixed N plant⁻¹ for the control (Table 4). Root irrigation with solutions containing thuricin 17 also increased nodule number for all three treatments, as compared with the control. T17-2 caused the greatest increase, at 21% more than the control. As with the leaf spray, thuricin 17 treatment did not affect nodule dry weight.

TABLE 4
Effects of thuricin 17 on soybean (cultivars OAC Oxford and
Korada) nodulation and nitrogen fixation (at final harvest). The
application method (leaf spray and root irrigation) and
concentration interaction means are shown.

| | Nodule | Nodule dry | N concentration | | Fixed N | | |
|---|---|---|---|---|---|---|---|
| | number | weight | Shoot | Root | Shoot | Root | Total |
| Treatment | plant⁻¹ | mg plant⁻¹ | mg g⁻¹ dw | | mg plant⁻¹ | | |
| Leaf spray | | | | | | | |
| Control^{a} | 88.1 d^{c} | 0.107 | 43.0 e | 29.1 | 35.16 b | 9.28 | 44.44 c |
| T17-1^{b} | 94.4 c | 0.112 | 44.0 de | 29.9 | 37.91 b | 9.75 | 47.66 c |
| T17-2 | 103.6 ab | 0.119 | 48.3 a | 30.4 | 45.58 a | 10.99 | 56.57 a |
| T17-3 | 101.1 b | 0.117 | 49.1 a | 31.1 | 45.51 a | 11.07 | 56.58 a |
| Root irrigation | | | | | | | |
| Control | 87.6 d | 0.107 | 42.8 e | 29.0 | 35.57 b | 9.16 | 44.73 c |
| T17-1 | 103.8 ab | 0.117 | 46.2 bcd | 29.8 | 42.51 a | 10.68 | 53.19 ab |
| T17-2 | 106.1 a | 0.117 | 47.0 abc | 30.4 | 42.05 a | 10.85 | 52.90 ab |
| T17-3 | 102.0 b | 0.113 | 45.2 cd | 29.9 | 37.93 b | 10.51 | 48.44b c |

^{a}Means were based on leaf spray treatments containing the surfactant Tween 20, while treatments for root irrigation did not.
^{b}T17-1, T17-2 and T17-3 represents a thuricin concentration of 5 × 10⁻¹¹, 5 × 10⁻¹⁰ and 5 × 10⁻⁹ M, respectively.
^{c}Means within the same column and factor followed by the same letter are not different (P ≦ 0.05) by an ANOVA-protected LSD test.
When letters are absent, ANOVA indicated no difference among means (n = 4).

Root irrigation with thuricin 17 solution also increased N concentration (mg g⁻¹ dry weight) in shoot tissue with T172 having the greatest effect (Table 4). As with the leaf spray, there was no difference among treatments for N concentration in root tissue. Both shoot and total fixed N per plant: were increased by T17-2, as compared to the control, whereas there was no difference for the amount of fixed N in root tissues. Values for shoot and total fixed N for T17-2 were 42.05 and 52.90 mg, respectively, while control values were 35.57 and 44.73 mg, respectively. Collectively these data show that the bacteriocin thuricin 17 directly enhances soybean growth.

### Example 4

### a) Production of Thuricin 17 (T17)

The Bacillus thuringiensis strain NEB17 was cultured in King's liquid medium at 25° C. on an orbital shaker for 48 h, rotating at 150 rev min⁻¹. The composition of this medium was as follows: protein peptone #3-20 g; K₂HPO₄−1.5 g; MgSO₄−0.75 g; glycerol—15 mL; distilled water−1000 mL. The entire culture was extracted by adding 0.4 volume of n-butanol. The butanol-water mixture had been shaken for 30 min and kept overnight at 4° C. The separated butanol phase was collected and evaporated at 450° C. using the rotary evaporator. The dried extract was resuspended in 20% acetonitrile and used for the purification of T17.

### (b) Purification of Thuricin 17

Butanol-soluble compounds, in 20% acetonitrile, were loaded on C18 solid phase cartridges and fractionated using 35% (acetonitrile:water, v/v), 43% and 100% acetonitrile. These fractions were collected. Aliquots of 0.2 mL were taken from them and used for the HPLC analyses to quantify T17 in fractions.

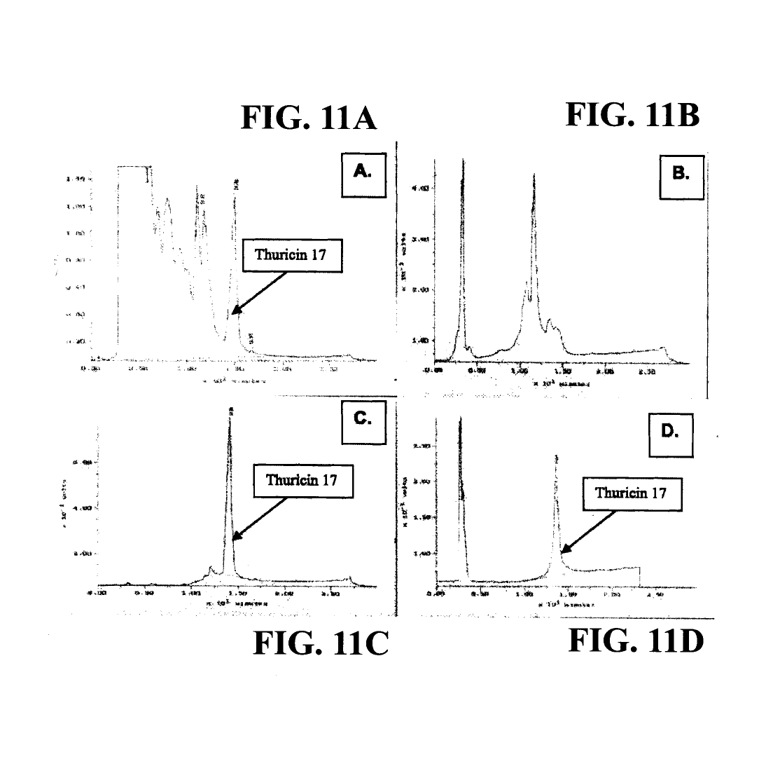

Two liters of bacterial culture of Bacillus thuringiensis strain NEB17 were extracted with 800 mL of n-butanol. The butanol-soluble material was evaporated and re-dissolved in 25 mL of 20% acetonitrile. The HPLC analysis showed the presence of thuricin 17 at a concentration of 5.42 mg mL⁻¹ in this solution (FIG. 11A). These 25 mL (containing 135.5 mg of bacteriocin) aliquots were loaded on PrepSep C18 cartridge. The retained compounds were eluted with 50 mL of 35, 43 and 100% acetonitrile. Thuricin 17 was not detected in the fraction with 35% acetonitrile (FIG. 11B) but was abundant (134.1 mg) in the fraction with 43% acetonitrile (FIG. 11C). Only 1.4 mg of Thuricin17 was present in the fraction with 100% acetonitrile (FIG. 11D). Multiple repetitions (10 times) of the procedure for purification of T17 showed that only 1.0±0.4% and 0.3±0.2% of the total loaded bacteriocin were eluted with 35% and 100% acetonitrile, respectively. The maximum of 98.7±0.3% was detected in fraction with 43% acetonitrile.

### Example 5

### Determination of T17 Promotion of Seedling Emergence and Earth Growth of Corn Supplied with and without Fertilizer (Hoagland's Solution)

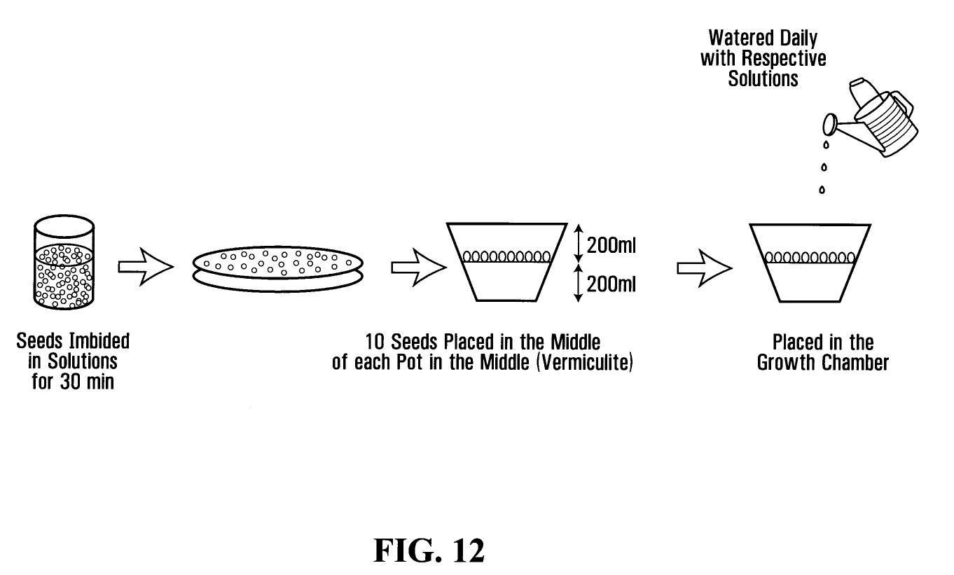

Seeds of corn (Zea mays hybrid var. MZ 310) were surface sterilized with 50% commercial bleach solution for 2-3 minutes and rinsed several times with distilled water (dH₂O). The seeds were then imbibed in the respective T17 (10⁻⁹, 10⁻¹⁰, 10⁻¹¹ M) or control (dH₂O) solutions for 30 minutes prior to transfer into individual Petri plates (FIG. 12). Ten seeds of corn were placed in previously surface sterilized 400 mL pots containing a Whatman™ filter paper (A4) and 200 mL of fine vermiculite. The seeds were watered with 100 mL of the respective T17 solution or dH₂O for the control and then covered with 200 mL of vermiculite. The seeds were given another 80 mL of the respective T17 solution or dH₂O. The pots were placed in a growth chamber under these conditions: 25/22° C. (day/night), 16 h photo period, and with a light intensity of 340 μmoles m⁻² s⁻¹. The study consisted of eight treatments of T17 concentrations of 10⁻⁹, 10⁻¹⁰, 10⁻¹¹ M dissolved in either dH₂O or Hoagland's solution (HS, ½ strength) and two controls (dH₂O and HS only).

In total, there were 40 pots with 5 pots per treatment. Corn plants were watered daily (50 mL) with their respective T17 solution without HS or dH₂O for the control. After 1 week of growth, the treatments of T17 solutions with HS were started. The control treatments were only given dH₂O or HS. Corn seedlings began to emerge after 3 days. Emergence for corn was considered when seedlings were 2 or 3 mm above the medium (FIG. 12). Plants were harvested after 14 days of growth. Data were collected on plant height and leaf area. Corn plants were separated into shoot and roots before oven drying at 60° C. for a minimum of 72 h, then measured for dry weight.

TABLE 5
Growth measurements of corn plants after 14 days of Thuricin
17 treatments with and without fertilizer (½ concentration of Hoagland's solution).
Values are means ± SE (in parentheses) of n = 4-5 replicates. T17 treatments without
fertilizer were supplied with distilled water only.

| | | Leaf area | Dry weight (g) | | |
|---|---|---|---|---|---|
| Treatment | Height (cm) | (cm²) | Shoot | Root | Total |

Without fertilizer

| | | | | | |
|---|---|---|---|---|---|
| Control | 18.8 (±0.9) | 21.6 (±1.4) | 0.11 (±0.01) | 0.20 (±0.01) | 0.31 (±0.01) |
| T17 10⁻⁹ M | 21.2 (±0.5) | 24.2 (±1.1) | 0.21 (±0.01) | 0.20 (±0.01) | 0.41 (±0.02) |
| T17 10⁻¹⁰ M | 20.0 (±0.5) | 21.6 (±1.4) | 0.24 (±0.01) | 0.20 (±0.01) | 0.44 (±0.02) |
| T17 10⁻¹¹ M | 19.7 (±0.6) | 18.1 (±1.8) | 0.14 (±0.01) | 0.19 (±0.01) | 0.33 (±0.02) |

With fertilizer

| | | | | | |
|---|---|---|---|---|---|
| Control | 25.9 (±0.6) | 34.7 (±2.5) | 0.24 (±0.02) | 0.23 (±0.00) | 0.48 (±0.02) |
| T17 10⁻⁹ M | 25.5 (±0.8) | 34.0 (±1.0) | 0.28 (±0.02) | 0.21 (±0.01) | 0.50 (±0.02) |
| T17 10⁻¹⁰ M | 25.9 (±0.5) | 33.7 (±0.3) | 0.36 (±0.01) | 0.24 (±0.01) | 0.60 (±0.02) |
| T17 10⁻¹⁰ M | 26.0 (±0.5) | 29.7 (±1.9) | 0.25 (±0.04) | 0.22 (±0.01) | 0.48 (±0.04) |

Corn treated with thuricin 17 solutions of 10⁻⁹, 10⁻¹⁰ and 10⁻¹¹ M had higher emergence rates from 72 to 80 h after seeding than the control plants, which were only given distilled water (FIG. 12). Furthermore, the higher emergence rates contributed to higher shoot and total plant dry weights of corn plants supplied with and without Hoagland's solution at 14 days of growth as compared to the control plants (Table 5).

### Example 6

### Determination of T17 Promotion of Seedling Emergence and Early Growth of Tomato Supplied with Fertilizer (Hoagland's Solution)

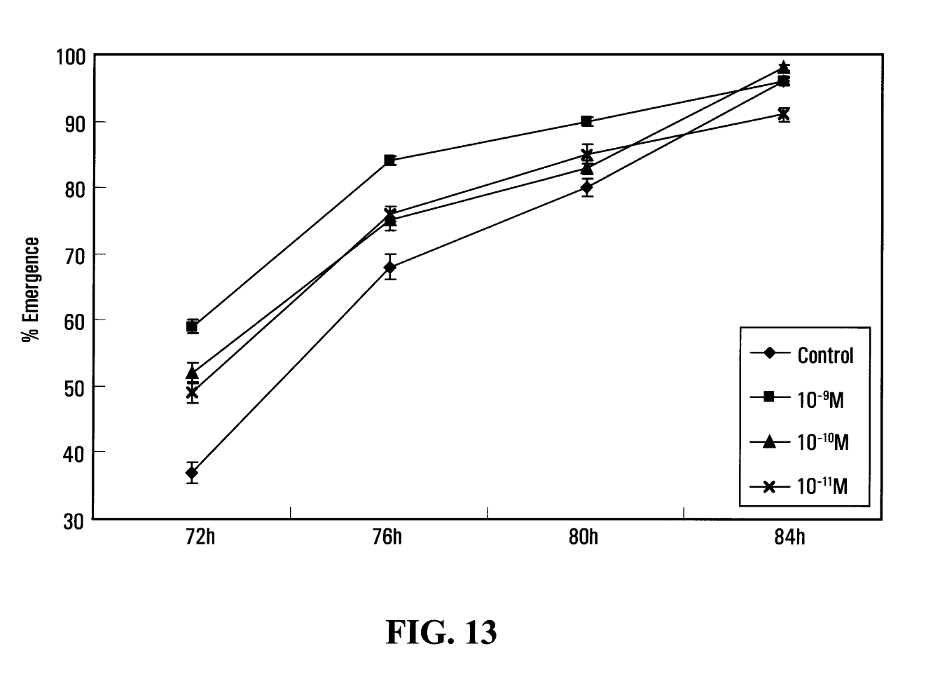

Seeds of tomato (Lycopersion esculentum L. F1 hybrid var. Veronica) were surface sterilized with 50% commercial bleach solution for 2-3 minutes and rinsed several times with distilled water (dH₂O). The seeds were then imbibed in the respective T17 (10⁻⁹, 10⁻¹⁰, 10⁻¹¹ M) dissolved in Hoagland's solution (HS, ½ strength) or control (HS) solutions for 30 minutes prior to transfer into individual Petri plates. Ten seeds of tomato were placed in previously surface sterilized 400 mL pots containing a Whatman filter paper (A4) and 400 mL of fine vermiculite. The seeds were watered with 180 mL of the respective T17 solution or HS for the control. The pots were placed in a growth chamber under these conditions: 25/22° C. (day/night), 16 h photoperiod, and with a light intensity of 340 μmoles m⁻² s⁻¹. In total, there were 20 pots with 5 pots per treatment. Tomato plants were watered daily (50 mL) with their respective T17 solution or HS. Tomato seedlings began to emerge after 4 days. Emergence for tomato was considered when seedlings were 2 or 3 mm above the medium (FIG. 13). Plants were harvested after 23 days of growth. Data were collected on plant height and leaf area. Tomato plants were separated into shoot and roots before oven drying at 60° C. for a minimum of 72 h, then measured for dry weight.

TABLE 6
Growth measurements of tomato plants after 23 days of T17
treatments with fertilizer (½ concentration of Hoagland's solution).
Values are means ± SE (in parentheses) of n = 4-5 replicates.

| | | Leaf area | Dry weight (g) | | |
|---|---|---|---|---|---|
| Treatment | Height (cm) | (cm²) | Shoot | Root | Total |
| Control | 18.6 (±0.3) | 42.6 (±1.3) | 0.25 (±0.02) | 0.08 (±0.00) | 0.33 (±0.02) |
| T17 10⁻⁹ M | 20.1 (±0.4) | 45.9 (±1.6) | 0.27 (±0.02) | 0.08 (±0.01) | 0.34 (±0.02) |
| T17 10⁻¹⁰ M | 20.2 (±0.1) | 56.9 (±0.8) | 0.30 (±0.01) | 0.08 (±0.00) | 0.38 (±0.01) |
| T17 10⁻¹¹ M | 20.2 (±0.6) | 48.4 (±0.9) | 0.29 (±0.02) | 0.08 (±0.00) | 0.37 (±0.02) |

Tomato plants showed a similar pattern to that of corn when supplied with Thuricin 17 solutions of 10⁻⁹, 10⁻¹⁰ and 10⁻¹¹ M. Tomato seeds treated with T17 solution of 10⁻⁹ M, had higher emergence rates from 96 to 144 h after seeding than the control plants, which were only given distilled water (FIG. 12). Yet at 23 days of growth, tomato plants treated with T17 10⁻⁹, 10⁻¹⁰ and 10⁻¹¹ M solutions had higher shoot and total plant dry weights than the control plants (Table 5).

### Example 7

### Determination of Bacthuricin F4 (BF4) Promotion of Seedling Emergence and Early Growth of Soy Bean

Seeds of soybean (Glycine max L. Merr. cv. OAC Bayfield) were surface sterilized with 400 mL L⁻¹ commercial bleach solution for 2-3 minutes and rinsed several times with distilled water (dH₂O). The seeds were then imbibed in the respective BF4 (10⁻⁹, 10⁻¹⁰, 10⁻¹¹ M) or control (dH₂O) solutions for 30 minutes prior to transfer into individual Petri plates. Ten seeds of soybean were placed in previously surface sterilized 400 mL pots containing a Whatman filter paper (A4) and 200 mL of fine vermiculite. The seeds were watered with 100 mL of the respective BF4 solution or dH₂O for the control and then covered with 200 mL of vermiculite. The seeds were given another 80 mL of the respective BF4 solution or dH₂O. The pots were placed in a growth chamber under these conditions: 25/22° C. (day/night), 16 h photoperiod, and with a light intensity of 340 μmoles m⁻² s⁻¹. In total, there were 20 pots with 5 pots per treatment. Soybean plants were watered daily (50 mL) with their respective BF4 solution or dH₂O for the control. Plants were harvested after 15 days of growth. Data were collected on plant height and leaf area. Soybean plants were separated into shoot and roots before oven drying at 80° C. for a minimum of 72 h, then measured for dry weight.

TABLE 7
Seedling emergence (%) after 96 h and growth measurements of
soybean plants after 15 days after Bacthuricin F4 treatments.
Values are means ± SE (in parentheses) of n = 3 replicates.

| | Seedling | | Leaf area | Dry weight (g) | |
|---|---|---|---|---|---|
| Treatment | emergence (%) | Height (cm) | (cm²) | Shoot | Root |
| Control | 64 (±2.4) | 13.0 (±0.3) | 42.5 (±3.0) | 0.24 (±0.01) | 0.08 (±0.01) |
| BF4 10⁻⁹ M | 58 (±4.9) | 14.0 (±0.7) | 44.5 (±2.7) | 0.24 (±0.01) | 0.09 (±0.01) |
| BF4 10⁻¹⁰ M | 62 (±7.3) | 13.4 (±0.1) | 39.5 (±2.5) | 0.26 (±0.01) | 0.09 (±0.03) |
| BF4 10⁻¹¹ M | 70 (±7.8) | 13.8 (±0.3) | 47.7 (±4.4) | 0.28 (±0.01) | 0.09 (±0.01) |

Soybean plants treated with BF4 at 10⁻¹⁰ and 10⁻¹¹ M had higher shoot dry weights at 15 days of growth as compared to the control plants.

### Example 8

### Determination of Isolated Bacteriocin (C85) Produced by Bacillus cereus UW85 on Promotion of Seedling Emergence and Early Growth of Soybean

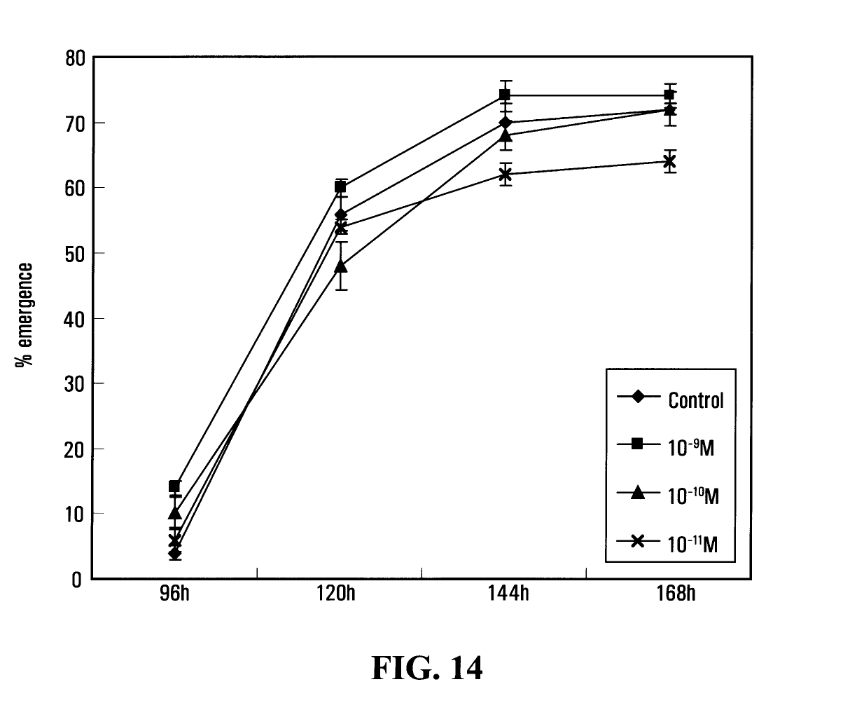

Seeds of soybean (Glycine max L. Merr. cv. OAC Bayfield) were surface sterilized with 400 mL L⁻¹ commercial bleach solution for 2-3 minutes and rinsed several times with distilled water (dH₂O). The seeds were then imbibed in the respective C85 (10⁻⁹, 10⁻¹⁰, 10⁻¹¹ M) or control (dH₂O) solutions for 30 minutes prior to transfer into individual Petri plates. Ten seeds of soybean were placed in previously surface sterilized 400 mL pots containing a Whatman filter paper (A4) and 200 mL of fine vermiculite. The seeds were watered with 100 mL of the respective C85 solution or dH₂O for the control and then covered with 200 mL of vermiculite. The seeds were given another 80 mL of the respective UW85 solution or dH₂O. The pots were placed in a growth chamber under these conditions: 25/22° C. (clay/night), 16 h photoperiod, and with a light intensity of 340 μmoles m⁻² s⁻¹. In total, there were 20 pots with 5 pots per treatment. Soybean plants were watered daily (50 mL) with their respective C85 solution or dH₂O for the control. Plants were harvested after 14 days of growth, and leaf area and shoot dry weight were measured. Soybean plants treated with the bacteriocin produced by Bacillus cereus UW85 at 10⁻⁹, 10⁻¹⁰ and 10⁻¹¹ M had higher leaf area and shoot dry weights than the control plants (FIG. 14).

### Example 9

### Effect of Chitin Hexamer and Thuricin 17 (T17) on Liginification-Related and Antioxidative Enzymes of Soybean Plant

### (a) Plant material

Soybean (Glycine max L. Merr. cv. OAC Bayfield) seeds were surface sterilized in 10% bleach, rinsed several times with distilled water and then germinated and grown in Vermiculite™ (Holiday, Montreal) in a growth chamber under a 16 h/8 h (day/night) regime (natural light supplemented with high pressure sodium lamps to reach the appropriate daylight), at 25±1° C., until they reached vegetative cotyledon (VC) stage (Fehr and Caviness, 1977).

### (b) Treatments

Treatments of chitin hexamer and thuricin 17 were applied when the seedling reached the first trifoliate stage (˜2 weeks old). Chitin hexamer and thuricin 17 treatments were applied through cut stems, as described by Orozco-Cardenas and Ryan (1999). The plants were excised at the base of the stem with a sharp scalpel and promptly placed in 2 mL Eppendorff™ tubes containing 0.5 mL of 100 μmol L⁻¹ chitin hexamer [(GlcNAc)₆], 0.5 mL of 1×10⁸ mol L⁻¹ thuricin 17, and chitin hexamer+thuricin 17 mixed (1:1) solution in phosphate buffer (15 mM sodium phosphate, pH 6.5). The control plants were treated with phosphate buffer solution alone. Once all the solution was taken up by the plants (4-6 h), they were immediately transferred to glass test tubes containing 20 mL distilled water. The plants were kept under constant white light (85 μmol·m⁻²·s⁻¹). Leaves were collected at 12, 24, 36, 48, 60 and 72 h after elicitor treatment, weighed, placed in plastic bags and stored immediately at −80° C.

### (c) Determination of PAL and TAL

Leaf samples (300 mg fresh weight) were extracted in 4 mL of buffer (50 mM Tris pH 8.5, 14.4 mmol L⁻¹ 2-mercaptoethanol, 1% w/v insoluble polyvinyl-polypyrrorolidone) and centrifuged at 6,000 g for 10 min at 4° C. The total protein concentration in soluble enzyme extracts was determined using the Bradford (1976) assay.

The method of Beaudoin-Eagan and Thorpe (1985) was used to estimate phenylalanine ammonia lyase (PAL) and tyrosine ammonia lyase (TAL) activities. The reaction mixture, at a final volume of 3 mL, consisted of 1.9 mL of 50 mM Tris-HCl buffer (pH 8.0), 100 μL of enzyme preparation and either 1.0 mL of 15 mM L-phenylalanine for PAL or 1.0 mL of 15 mM L-tyrosine for TAL. The assay was started by the addition of enzyme extract after an initial incubation for 60 min at 40° C. The reactions were stopped by the addition of 200 μL of 6 N HCl. The amounts of trans-cinnamic and p-coumaric acids formed were determined by measuring absorbance at 290 and 330 nm, respectively, against an identical mixture in which D-phenylalanine was substituted for L-phenylalanine and D-tyrosine for L-tyrosine. The enzyme activity was expressed in nmoles (cinnamic or coumaric acid) mg protein⁻¹ min⁻¹, where 1 unit is defined as 1 mmoles (cinnamic or coumaric acid) mg protein⁻¹ min⁻¹.

### (d) Determination of Total Phenolics

Total phenolic content was determined by the Folin-Ciocalteu method (Singleton and Rossi, 1965). The assay mixture contained 50 μL of sample with 0.475 mL of 0.25 N Folin-Ciocalteu reagent (Sigma Chemical Co.). After 3 min, 0.475 mL of 1 mol L⁻¹ Na₂CO₃ was added and after 1 h absorbance was measured. The phenolic contents were estimated using a standard curve prepared with gallic acid. The total phenolic content was expressed as gallic acid equivalents (GAE) in mg g⁻¹ fresh weight (FW).

### (e) Determination of POD and SOD Activities

The activity of peroxidase (POD) was assessed using the method of Chance and Maehly (1955). The reaction mixture consisted with 50 μL of 20 mM guaiacol, 2.8 mL of 50 mM Tris-HCl buffer (pH 8.0) and 0.1 mL extract. The reaction was started with addition of 20 μL of 40 mM H₂O₂ and the change in the absorbance at 470 nm was recorded for 1 min. The activity of peroxidase was calculated using an extinction coefficient for the tetraguaiacol of 26.6 mM⁻¹ cm⁻¹ at 470 nm. One unit of enzymatic activity was defined as the amount of enzyme required for the formation of 1 μmol of tetraguaiacol per minute.

The activity of superoxide dismutase (SOD) was determined by measuring its ability to inhibit the photoreduction of nitroblue tetrazolium (NBT) following the method of Giannopolitis and Ries (1977). The reaction mixture (3.0 mL) consisted of 63 μM NBT (nitroblue tetrazolium), 1.3 μM riboflavin, 13 mM methionine, 0.1 mM EDTA, 50 mM Tris-HCl (pH 8.0), and 50 μL extract. The mixture was held in a test tube and placed for 20 min under light at 78 μmol photons s⁻¹ m⁻². Absorbance was recorded at 560 nm. A non-illuminated reaction mixture that did not develop color served as the control, and its absorbance was subtracted from the A₅₆₀ of the reaction solution. One unit of enzyme activity was defined as the amount of enzyme required to inhibit 50% of the NBT photoreduction, in comparison with tubes lacking the plant extract.

### (f) Detection of Antioxidant Enzymes

For active staining of POD after separation through 12.5% polyacrylamide gel electrophoresis (PAGE), the gels was soaked for 10 min in 50 mM Tris buffer (pH 8.0) then incubated with 0.46% (v/v) guaiacol, and 13 mM H₂O₂ in the same buffer at room temperature until red bands appeared; these were subsequently fixed in water/methanol/acetic acid (6.5:2.5:1, v/v/v) (Caruso et al., 1999).

For the catalase activity (CAT) staining after 12.5% PAGE, the gel was incubated with 3.2 mM H₂O₂ for 20 min, and a treatment with a solution containing 1% FeCl₃ and 1% K₃Fe(CN)₆ for 10 min, as described by Racchi et al (2001).

SOD activity staining after 12.5% PAGE, was performed to determine any change in the activity of SOD isozymes. The gel was soaked in 50 mM Tris-HCl (pH 8.0) containing 2.5 mM NBT for 25 min at room temperature. Cu/Zn-SODs were inhibited with KCN and H₂O₂ and Fe-SODs were inhibited with H₂O₂; Mn-SODs are resistant to both inhibitors (Fridovich, 1989). The gel was rinsed in distilled water and then incubated in the same buffer, containing 28 mM TEMED and 28 μM riboflavin, for 30 min. The gel was placed under an illuminator for 30 min to develop the purple color, except for the areas where SOD was localized in gel.

Chitin hexamer elicited increases in PAL, TAL, total phenolic compounds, POD and CAT but SOD activity was not induced. Thuricin 17 elicited PAL, TAL, total phenolic compounds, POD and SOD, but CAT activity was not induced.

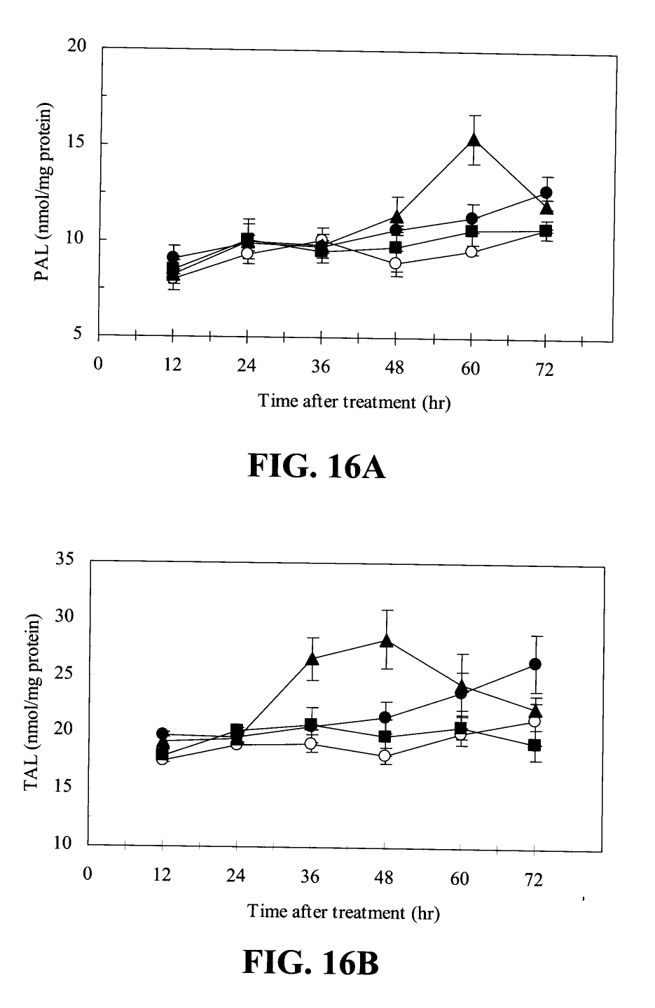

Changes in lignification related enzymes were apparent by 72 h after chitin hexamer and/or thuricin 17 treatment of soybean leaves (FIG. 16). PAL activity in T17 treated leaves increased until 60 h after treatment and thereafter decreased (FIG. 16A). PAL activity in chitin hexamer treated leaves increased continuously throughout experiment period, while PAL in chitin hexamer and thuricin 17 treated leaves did not increase above the control level. At 60 h, PAL activity increased by 61.8% in thuricin 17 treated leaves and 8.4% in chitin hexamer treated leaves, compared with control. At 72 h, PAL activity was 11.5 and 18.1%, respectively, greater than the control in T17 and chitin hexamer treated leaves. Vander et al. (1998) found that chitin oligomers (degree of polymerization 4-10) did not elicit PAL activities at 24 h after injection into intercellular spaces of wheat leaves whereas, deacetylation levels of 35, 50 and 60% were determined, indicating PAL induction. Fully deacetylated chitooligosaccharides (chitosan oligomers) induce, depending on their degree of polymerization and concentration, PAL activation in Arabidopsis thaliana cell suspensions whereas reacetylation of the chitosan oligomer elicitors did not affect the activation of PAL (Cabrera, 2006).

TAL activity in T17 treated leaves increased until 48 h after treatment and thereafter slightly decreased (FIG. 16B). TAL activity in chitin hexaamer treated leaves increased continuously throughout experiment period, while TAL levels in chitin hexamer and T17 treated leaves were unaffected by treatment and remained low. At 48 h, TAL activity was increased by 57.0% in T17 treated leaves but by only 18.8% in chitin hexamer treated leaves, as compared with the control treatment. At 72 h, TAL activity was increased by 5.0% in T17 and 23.8% in leaves of chitin hexamer treated plants, respectively, compared with the control.

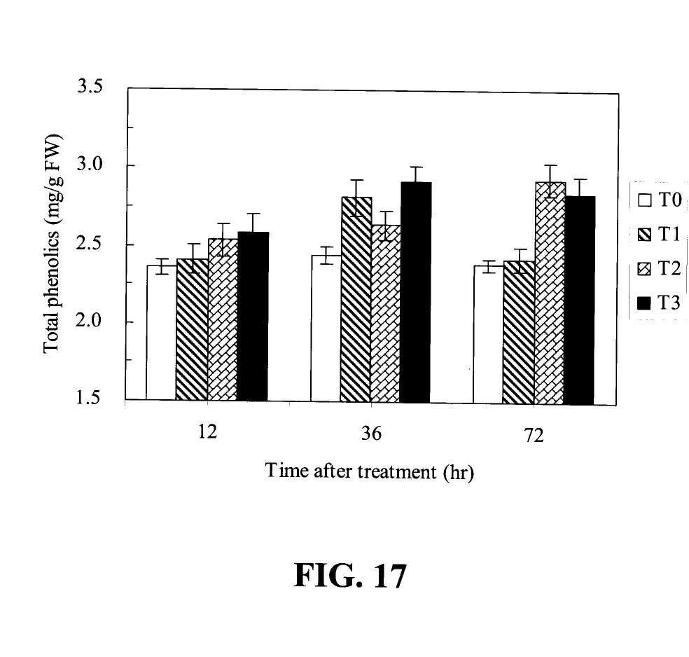

The concentration of total phenolic compounds in soybean leaves was determined at 12, 36 and 72 h after chitin hexamer and T17 treatments (FIG. 17). At 36 h, total phenolics increased by 15.3% in chitin hexamer treated leaves, by 8.0% following T17 treatment, and by 19.3% in chitin hexamer and T17 treated leaves, compared with the control. At 72 h, total phenolics increased by 23.2% in T17 treated leaves and by 19.0% in chitin hexamer and T17 treated leaves, but by only 1.4% in chitin hexamer treated leaves, as compared with the control. Treatment of insoluble mycelial walls of a fungus, Chaetomium globosum, stimulated the induction of PAL and the accumulation of phenolic acids in cultured carrot cells (Kurosaki et al., 1986). Chitin and chitosan have been shown to be effective elicitors in the hypersensitive lignification response of intact (Vander et al., 1998) and wounded (Barber et al., 1989; Pearce and Ride, 1982) plants. Also, the elicitation of lignification-related enzyme activity not only depends on the chain length but also on the abundance of the chitin oligomers (Pearce and Ride, 1982).

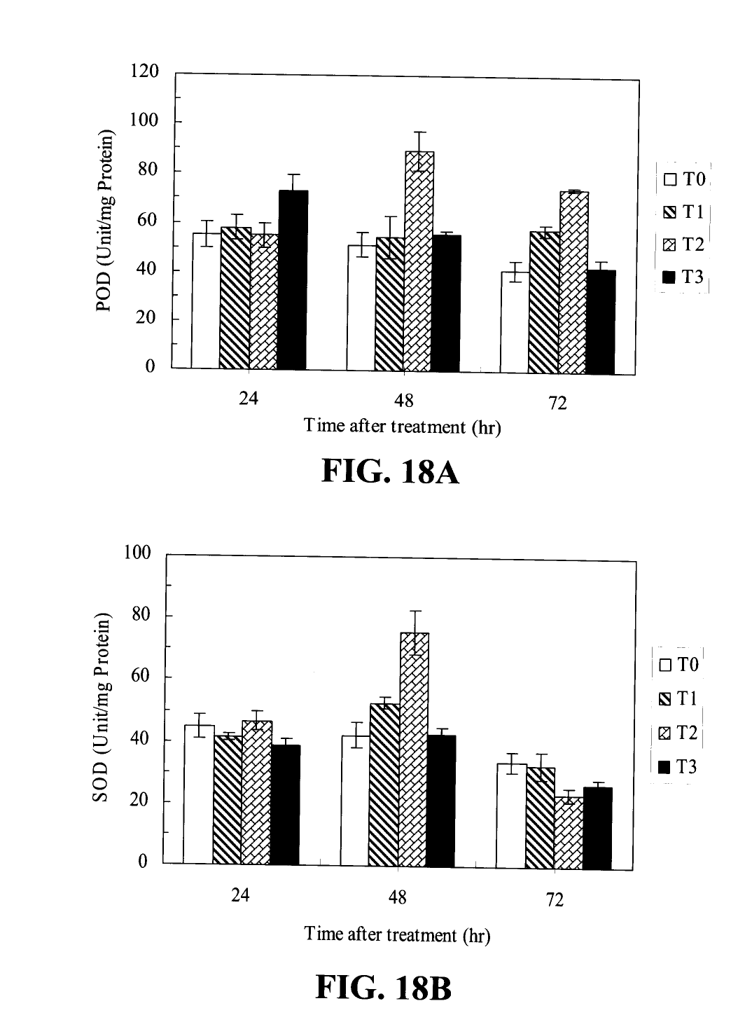

POD and SOD activity in soybean leaves was measured at 24, 48 and 72 h after chitin hexamer and T17 treatment (FIG. 18). At 24 h, POD activity increased by 31.9% in chitin hexamer and T17 treated leaves (FIG. 18A). At 48 h, POD activity was increased by 74.6% in T17 treated leaves. At 72 h, POD activity increased by 40.3% in chitin hexamer and by 81.2% in T17, but by only 3.4% in chitin hexamer and T17 treated leaves, compared with control leaves. At 48 h, SOD activity increased by 24.9% in chitin hexamer and by 79.9% in T17 treated leaves, compared with control leaves (FIG. 18B). Chitin oligomers (degree of polymerization 7-10) induced POD activities at 24 h after injection into intercellular spaces of wheat leaves whereas POD induction increased dramatically with DAs of 50 and 60% (Vandler et al., 1998).

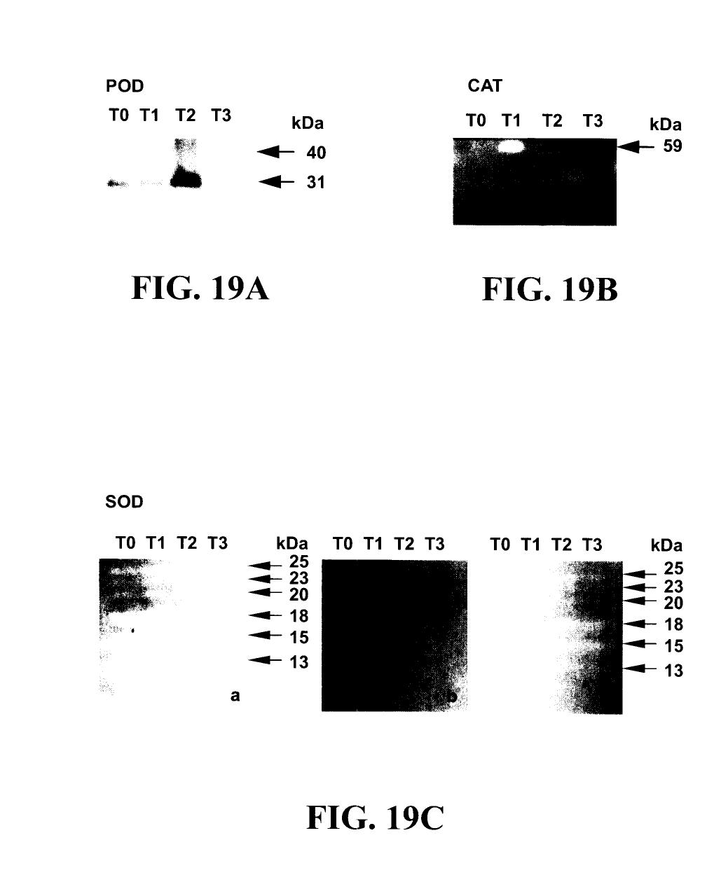

After polyacryamide gel electrohoresis (PAGE) activities of POD, CAT and SOD were measured to detect possible changes in isozyme levels of soybean leaves (FIG. 19). At 60 h, two bands (40 and 31 kDa) stained for POD activity in leaves treated with thuricin 17 (FIG. 19A). Activity of the 31 kDa isoenzyme was induced stronger in T17 treated leaves than control treatment. One band (59 kDa) from leaves treated with the chitin hexamer stained for CAT activity (FIG. 19B). The electrophoretic pattern of SODs in leaves showed six bands (25, 23, 20, 18, 15 and 13 kDa) of activity, which were identified as Fe-SODs, since they were inhibited by H₂O₂ (FIG. 19C(b)) and were activated by KCN (FIG. 19C(c)). Two major Fe-SOD bands of these were induced stronger in leaves treated with T17 and chitin hexamer+T17 than control treatment (FIG. 19C(a)). Plants generally contain Fe-SOD and Cu/Zn-SOD in chloroplasts, Cu/Zn-SOD in the cytosol and Mn-SOD in the mitochondrial matrix and proxisomes (Bower et al., 1994). An increase in peroxisomal Mn-SOD activity has been reported to occur under stress as a specific defense against oxidative stress in pea plants (Palma et al., 1987).

### LIST OF REFERENCES

Abd-Alla, M H, Regulation of nodule formation in soybean-Bradyrhizobium symbiosis is controlled by shoot or/and root signals, Plant Growth Regulation, 34:241-250, 2001.
Achouak, W. et al., Comparative phylogeny of rrs and nifH genes in the Bacillaceae, International Journal of Systematic Bacteriology, 49:961-967, 1999.
Agata, N. et al., A novel dodecadipeptide, cereulide, is an emetic toxin of Bacillus cereus, FEBS Microbiology Letters, 129:17-20, 1995.
Agrawal A A, Overcompensation of plants in response to herbivory and the by-product benefits of mutualism, Trends in Plant Science, 5:309-313, 2000.
Aguilera, M. et al., Paenibacillus jamilae sp. nov., an exopolysaccharide-producing bacterium able to grow in olive-mill wastewater, International Journal of Systematic and Evolutionary Microbiology, 51:1687-1692, 2001.
Ahern, M. et al., Isolation and characterization of a novel bacteriocin produced by Bacillus thuringiensis strain B439, FEMS Microbiology Letters, 220:127-131, 2003.
Andrews, R. E. et al., Protease activation of the entomocidal protoxin of Bacillus thuringiensis subsp. kurstaki, Applied and Environmental Microbiology, 50:737-742, 1985.
Apostol, I. et al., Rapid stimulation of an oxidative burst during elicitation of cultured plant cells: Role in defense and signal transduction, Plant Physiology, 90:109-116, 1989.
Arlat, M. et al., PopA1, a protein which induces a hypersensitivity-like response on specific Petunia genotypes, is secreted via the Hrp pathway of Pseudomonas solanacearum, EMBO Journal, 13:543-553, 1994.
Aronson, A. I. et al., Structure and morphogenesis of the bacterial spore coat, Bacteriology Reviews, 40:360-402, 1976.
Asghar, H. N. et al., Relationship between in vitro production of auxin and their growth-promoting activities in Brassica juncea L, Biology and Fertility in Soils, 35:231-237, 2002.

Atlas R M, Handbook of media for environmental microbiology, CRC Press, Boca Raton, Fla., USA, 1995.
Ayers, A. R. et al., Host-pathogen interactions: IX. Quantitative assays of elicitor activity and characterization of the elicitor present in the extracellular medium of cultures of Phytophthora megasperma var. sojae, Plant Physiology, 57:751-759, 1976.
Bacilio-Jimenez, M. et al., Chemical characterization of root exudates from rice (Oryza sativa L.) and their effects on the chemotactic response of endophytic bacteria, Plant and Soil, 249:271-277, 2003.
Bai Y. et al., Isolation of plant-growth-promoting Bacillus strains from soybean root nodules, Canadian Journal of Microbiology, 48:230-238, 2002.
Bai Y. et al., Enhanced soybean plant growth resulting from co-inoculation of Bacillus strains with Bradyrhizobium japonicum, Crop Science, 43:1774-1781, 2003.
Bai, Y. et al., An inducible activator produced by Serratia proteomaculans strain and its soybean growth-promoting activity under greenhouse conditions, Journal of Experimental Botany, 53:1495-1502, 2002.
Barber, M. S. et al, Chitin oligosaccharides elicit lignification in wounded wheat leaves, Physiology Molecular Plant Pathology, 34:3-12, 1989.
Bastian F. et al., Production of indole-3-acetic acid and gibberellins A₁ and A₃ by Acetobacter diazotrophicus and Herbaspirillum seropedicae in chemically-defined culture media, Plant Growth Regulation, 24:7-11, 1998.
Beaudoin-Eagan et al., Tyrosine and phenylalanine ammonia lyase activities during shoot initiation in tobacco callus cultures, Plant Physiology, 78:438-441, 1985.
Ben-Shushan, G. et al., Two different propionicins produced by Propionibacterium thoenii P-127, Peptides, 24:1733-1740, 2003.
Bhuvaneawari T V et al., Early events in the infection of soybean [Glycine max (L.) Merr.] by Rhizobium japonicum I. location of infectible root cells, Plant Physiology, 66:1027-1031, 1980.
Bizani, D. et al., Characterization of a bacteriocin produced by a newly isolated Bacillus sp. Strain 8A, Journal of Applied Microbiology, 83:512-519, 2002.
Bottini, R. et al., Gibberellin production by bacteria and its involvement in plant growth promotion and yield increase, Applied Microbiology and Biotechnology, 65:497-503, 2004.
Bower, C. et al., Superoxide dismutage in plants, Critical Reviews in Plant Science, 13:199-218, 1994.
Bradford M M, A rapid and sensitive for the quantitation of microgram quantities of protein utilizing the principle of protein-dye binding, Annals of Biochemistry, 72:248-254, 1976.
Bradford, M. M., A rapid and sensitive method for the quantification of microgram quantities of protein utilizing the principle of protein-dye binding, Analytical Biochemistry, 72:1151-1154, 1976.
Bragg, T. S. et al., Nucleotide sequence and analysis of the lethal factor gene (lef) from Bacillus anthracis. Gene 44, 71-78, 1989.
Breitweiser, A. et al., Evidence for an S-layer protein pool in the peptidoglycan of Bacillus stearothermophilus, Journal of Bacteriology, 174:8008-8015, 1992.
Bringhurst, R. M. et al., Galactosides in the rhizosphere: utilization by Sinorhizobium meliloti and development of a biosensor, Proceedings of the National Academy of the United States, 98:4540-4545, 2001.
Broughton, W. J. et al., Signals exchanged between legumes and Rhizobium: agricultural uses and perspectives, Plant and Soil, 252:129-137, 2003.
Brown, E. W. et al., Phylogenetic relationships of necrogeneic Erwinia and Brenneria species as revealed by blyceraldehyde-3-phosphate dehydrogenase gene sequences, International Journal of Systematic Evolution and Microbiology, 50:2057-2068, 2000.
Cabrera, J. C. et al., Size, acetylation and concentration of chitooligosaccharide elicitors determine the switch from defense involving PAL activation to cell death and water peroxide production in Arabidopsis cell suspensions, Physiology Plant, 127:44-56, 2006.
Caetano-Anolles G. et al., Plant genetic control of nodulation, Annual Review of Microbiology, 45:345-382, 1991.
Camacho, M. et al., Co-inoculation with Bacillus sp. CECT450 improves nodulation in Phaseolus vulgaris L, Canadian Journal of Microbiology, 47:1058-1062, 2001.
Carroll, J. et al., Analysis of Bacillus thuringiensis d-endotoxin action on insect-membrane permeability using a light-scattering assay, European Journal of Biochemistry, 214:771-778, 1993.
Carson K. C. et al., Hydroxamate siderophores of root nodule bacteria, Soil Biology and Biochemistry, 32:11-21, 2000.
Caruso, C. et al., Induction of pathogenesis-related proteins in germinating wheat seeds infected with Fusarium culmorum, Plant Science, 140:107-120, 1999.
Cha, C. et al., Production of acyl-homoserine lactone quorum-sensing signals by Gram-negative plant-associated bacteria, Molecular Plant and Microbe Interactions, 11:1119-1129, 1998.
Chada, V. G. R. et al, Morphogenesis of Bacillus spore surfaces, Journal of Bacteriology, 185:6255-6261, 2003.
Chance, B. et al., Assay of catalase and peroxidase: In Methods in Enzymology, Ed. S. P. Colowick, N. O. Kaplan, Academic Press, New York, 764-775, 1955.
Chen H and Hoover D G 2003. Bacteriocins and their Food Applications. Comprehensive Reviews in Food Science and Food Safety 2: 82-100.
Cherif A. et al., Detection and characterization of the novel bacteriocin entomocin 9, and safety evaluation of its producer, Bacillus thuringiensis ssp. entomocidus HD9, Journal of Applied Microbiology, 95:990-1000, 2003.
Cherif A. et al., Thuricin 7: a novel bacteriocin produced by Bacillus thuringiensis BMG1.7, a new strain isolated from soil, Letters in Applied Microbiology, 32:243-247, 2001.

Choma C. et al., The enterotoxin T (BcET) from Bacillus cereus can probably not contribute to food poisoning, FEMS Microbiology Letters, 19:115-119, 2002.
Conrath, U. et al., Chitosan-elicited synthesis of callose and of coumarin derivatives in parsley cell suspension cultures, Plant Cell Reporter, 8:152-55, 1989.
Côté, F. et al., Oligosaccharins: Structures and signal transduction, Plant Molecular Biology, 26:1379-1411, 1994.
Daffonchio, D. et al., Homoduplex and heteroduplex polymorphisms of the amplified ribosomal 16S-23S internal transcribed spacers describe genetic relationships in the “Bacillus cereus group”, Applied and Environmental Microbiology, 66:5460-5468, 2000.
Dashti, N. et al, Application of plant growth-promoting rhizobacteria to soybean (Glycine max (L.) Merr.) increases protein and dry matter yield under short-season conditions, Plant and Soil, 188:33-41, 1997.
Degrassi, G. et al., Plant growth-promoting Pseudomonas putida WCS358 produces and secretes four cyclic dipeptides: cross-talk with quorum sensing bacterial sensors, Current Microbiology, 45:250-254, 2002.
Dixon, R. A. et al., Phytoalexins: enzymology and molecular biology, Advance Enzymology, 55:1-136, 1983.
Dong, Y. H. et al., Insecticidal Bacillus thuringiensis silences Erwinia carotovora virulence by a new form of microbial antagonism, signal interference, Applied and Environmental Microbiology, 70:954-960, 2004.
Driks, A., Maximum shields: the armor plating of the bacterial spore, Trends in Microbiology, 10:251-254, 2002.
Driscoll, B. T. et al., A novel bacteriocin, thuricin 17, produced by PGPR strain Bacillus thuringiensis NEB17: isolation and classification, Journal of Applied Microbiology, 100:545-554, 2006.
Dubnau, D., Genetic competence in B. subtilis, Microbiology Reviews, 55:395-424, 1991.
Ebel, J. et al., Elicitors of plant defense responses, International Review Cytology, 148:-36, 1994.
Eijsink V G H, Axelsson L, Diep D B, Håavarstein L S, Holo H and Nes I F 2002. Production of class II bacteriocins by lactic acid bacteria; an example of biological warfare and communication. Antonie van Leeuwenhoek 81: 639-654, 2002.
Ejiofor, A. O. et al., Physiological and molecular detection of crystalliferous Bacillus thuringiensis strains from habitats in the South Central United States, Journal of Industrial Microbiology and Biotechnology, 28:284-290, 2002.
Errington, J., Bacillus subtilis sporulation: regulation of gene expression and control of morphogenesis, Microbiological Reviews, 57:1-33, 1993.
Estruch, J. J. et al., Vip3A, a novel Bacillus thuringiensis vegetative insecticidal protein with a wide spectrum of activities against lepidopteron insects, Proceedings of the National Academy of Sciences USA, 93:5389-5394, 1996.
Favret, M. E. et al., Thuricin: the bacteriocin produced by Bacillus thuringiensis, Journal of Invertebrate Pathology, 53:206-216, 1989.
Fehr W. R. et al., Stage of development descriptions for soybeans, Glycine max (L.) Merrill, Crop Science, 11:929-931, 1971.
Fehr, W. R. et al., Stages of soybean development, Special Report, Agriculture and Home Economics Experiment Station, Iowa State University, 80:11, 1977.
Fray, R. G., Altering plant-microbe interaction through artificially manipulating bacterial quorum sensing, Annals of Botany, 89:245-253, 2002.
Fridovich, Superoxide dismutase. an adaptation to a paramagnetic gas, Journal of Biological Chemistry, 264:7761-7764, 1989.
Gardner, I. C. et al., Mycorrhizal improvement in non-leguminous nitrogen fixing associations with particular reference to Hippophae rhamnoides L, Plant and Soil, 78:189-199, 1984.
Giannopolitis, C. N. et al., Superoxide Dismutases: I. Occurrence in Higher Plants, Plant Physiology, 59:309-314, 1977.
Gilbert, R. J. et al., Bacillus cereus enterotoxins: present status, Biochemical Society Transactions, 12:198-200, 1984.
Gill, S. S. et al., The mode of action of Bacillus thuringiensis endotoxins, Annual Review of Entomology, 37:615-636, 1992.
Glick, B. R., The enhancement of plant growth by free-living bacteria, Canadian Journal of Microbiology, 41:109-117, 1995.
Gonzalez-Lopez, J. et al., Production of auxins, gibberellins and cytokinins by Azotobacter vinelandii ATCC 12837 in chemically-defined media and dialyzed soil media, Soil Biology and Biochemistry, 18:119-120, 1986.
Gray E. J. et al., Intracellular and Extracellular PGPR: Commonalties and distinctions in the plant-bacterium signaling processes, Soil Biology and Biochemistry, 37:395-412, 2005.
Jack, R. W et al., Bacteriocins of Gram-positive bacteria, Microbiological Reviews, 59:171-200, 1995.
Gray, E. J. et al., A novel bacteriocin, thuricin 17, produced by plant growth promoting rhizobacteria strain Bacillus thuringiensis NEB17: isolation and classification, Journal of Applied Microbiology, 100:545-554, 2006a.
Gray, E. J. et al., Proteomic analysis of the bacteriocin, thuricin 17 produced by Bacillus thuringiensis NEB17, FEMS Microbiology letters, 255:27-32, 2006b.
Gray, K. M., Intercellular communication and group behavior in bacteria, Trends in Microbiology, 5:184-188, 1997.
Grossman, A. D., Genetic networks controlling the initiation of sporulation and the development of competence in Bacillus subtilis, Annals Review of Genetics, 29:477-508, 1995.
Guidi-Rontani, C. et al., Fate of germinated Bacillus anthracis spores in primary murine macrophages, Microbiology, 42:931-938, 2001.
Gutierrez Manero, F. J. et al., The influence of native rhizobacteria on European alder (Alnus glutinosa L. Gaertn.) growth. II. Characterization and biological assays of metabolites from growth promoting and growth inhibiting bacteria, Plant and Soil, 182:67-74, 1996.
Gutierrez Manero, F. J. et al., The plant-growth-promoting rhizobacteria Bacillus pumilus and Bacillus licheniformis produce high amounts of physiologically active gibberellins, Physiologia Plantarum, 111:206-211, 2001.
Gyobu, Y. et al., Proposal to transfer Actinomadura carminata to a new subspecies of the genus Nonomuraea as Nonomueraea roseoviolaceae subsp. Carminata comb. nov, International Journal of Systematic Evolution and Microbiology, 51:881-889, 2001.
Hagen, G., The control of gene expression by auxin. In: Davies, P. J., ed. Plant Hormones and Their Role in Plant Growth and Development. Kluwer Academic Publishers, Dordrecht, The Netherlands, 149-163, 1990.
Hallmann, J. et al., Bacterial endophytes in agricultural crops, Canadian Journal of Microbiology, 43:895-914, 1997.
Hansen, B. M. et al., Detection of enterotoxic strains of B. cereus and B. thuringiensis by PCR analysis, Applied and Environmental Microbiology, 67:185-189, 2001.
Hansen, J. N., Nisin as a model food preservative, Critical Reviews in Food Science and Nutrition, 34:69-93, 1994.
Helgason, E. et al., Bacillus anthracis, Bacillus cereus, and Bacillus thuringiensis—one species on the basis of genetic evidence, Applied and Environmental Microbiology, 66:2627-2630, 2000.
Helgason, E. et al., Multilocus sequence typing for bacteria of the Bacillus cereus group, Applied and Environmental Microbiology, 70(1):191-201, 2004.
Hill, K. K. et al., Fluorescent amplified length fragment polymorphisms analysis of Bacillus anthracis, Bacillus cereus and Bacillus thuringiensis isolates, Applied Environmental Microbiology, 70:1068-1080, 2004.
Horsburgh, M. et al., Transcriptional responses during outgrowth of Bacillus subtlis endospores, Microbiology, 147:2933-2941, 2001.
Hyronimus, B. et al., Coagulin, a bacteriocin-like inhibitory substance produced by Bacillus coagulans I₄, Journal of Applied Microbiology, 85:42-50, 1998.
Jack W. R. et al., Bacteriocins of Gram-positive bacteria, Microbiology Reviews, 59:171-200, 1995.
Jameson P., Cytokinins and auxins in plant-pathogen interactions—an overview, Plant Growth Regulation, 32:369-380, 2000.
Jensen, G. B. et al., The hidden lifestyles of Bacillus cereus and relatives, Environmental Microbiology, 5:631-640, 2003.
Jimenez-Diaz, R. et al., Purification and partial amino acid sequence of Plantaricin S, a bacteriocin produced by Lactobacillus plantarum LPCO10, the activity of which depends on the complementary action of two peptides, Applied and Environmental Microbiology, 61:4459-4463, 1995.
Johnson, G. V. et al., Reduction and transport of Fe from siderophores, Plant and Soil, 241:27-33, 2002.
Kamoun F. et al., Purification, amino acid sequence and characterization of Bacthuricin F4, a new bacteriocins produced by Bacillus thuringiensis, Journal of Applied Microbiology, 98:881-888, 2005.
Karakas Sen, A. et al., Post-translational modification of Nisin: the involvement of NisB in the dehydration process, Europen Journal of Biochemistry, 261:524-532, 1999.
Kaur, K. et al., Dynamic relationships among Type IIa bacteriocins: temperature effects on antimicrobial activity and on structure of the C-terminal amphipathic α-helix as a receptor-binding region, Biochemistry, 43:9009-9020, 2004.
Kim, W. et al., Genetic relationships of Bacillus anthracis and closely related species based on variable-number tandem repeat analysis and BOX-PCR genomic fingerprinting, FEMS Microbiology Letters, 207:21-27, 2001.
Kimura, H. et al., Novel bacteriocin of Pediococcus sp. ISK-1 isolated from well-aged bed of fermented rice bran, Annals of the New York Academy of Sciences, 864:345-348, 1998.
Kinsinger, R. F. et al., Notes: rapid surface motility in Bacillus subtilis is dependent on extracellular surfactin and potassium ion, Journal of Bacteriology, 183:5627-5631, 2003.
Klaenhammer, T. R., Genetics of bacteriocins produced by lactic acid bacteria, FEMS Microbiology Reviews, 12:39-86, 1993.
Klein, C. et al., Analysis of genes involved in biosynthesis of the lantibiotic subtilin, Applied and Environmental Microbiology, 58:1795-1802, 1992.
Kloepper J. W. et al., Enhanced plant growth by siderophores produced by plant growth-promoting rhizobacteria, Nature, 286:885-8, 36, 1980.
Korem, M. et al., Characterization of RAP, a quorum sensing activator of Staphylococcus aureus, FEMS Microbiology Letters, 223:167-175, 2003.
Kurosaki, F. et al., Induction of chitinase and phenylalanine ammonia-lyase in culture carrot cells trated with fingsl mycelial wall, Plant Cell Physiology, 27:1587-1591, 1986.
Lai, E. M. et al., Proteomic analysis of the spore coats of Bacillus subtilis and Bacillus anthracis, Journal of Bacteriology, 185:1443-1454, 2003.
Le Marrec, C. et al., Biochemical and genetic characterization of coagulin, a new antilisterial bacteriocin in the pediocin family of bacteriocins, produced by Bacillus coagulans I₄, Applied and Environmental Microbiology, 66:5213-5220, 2000.
Lee, K. H. et al., Partial characterization of polyfermenticin SCD, a newly identified bacteriocin of Bacillus polyfermenticus, Letters in Applied Microbiology, 32:146-151, 2001.

Lee, N. K. et al., Partial characterization of lacticin NK24, a newly identified bacteriocin from Lactococcus lactis NK24 isolated front jeot-gal, Food Microbiology, 18:17-24, 2001.
Lee, S. J. et al., Genes encoding the N-acyl homoserine lactone degrading enzyme are widespread in many subspecies of Bacillus thuringiensis, Applied and Environment Microbiology, 68:3919-3924, 2002.
Lian, B. et al., Evidence for the production of chemical compounds analogous to Nod factor by the silicate bacterium Bacillus circulans GY92, Microbial Research, 156:289-292, 2001.
Lithgow, J. K. et al, The regulatory locus cinR1 in Rhizobium leguminosarum controls a network of quorum-sensing loci, Molecular Microbiology, 37:81-97, 2000.
Liu, H. et al., Formation and composition of the Bacillus anthracis endospore, Journal of Bacteriology, 186:164-178, 2004.
Lodewyckx, C. et al., Endophytic bacteria and their potential applications, Critical Reviews in Plant Science, 21:583-606, 2002.
Loh, J. et al., Quorum sensing in plant-associated bacteria. Current Opinions in Plant Biology 5, 1-6, 2002.
Loh, J. et al., Population density-dependent regulation of the Bradyrhizobium japonicum nodulation genes, Molecular Microbiology, 42:3746, 2001.
Mahaffee, W. F. et al., Temporal changes in the bacterial communities of soil, rhizosphere, and endorhiza associated with field-grown cucumber (Cucumis sativus L.), Microbial Ecology, 34:210-223, 1997.
Martinez-Romero, E. et al., Sesbania herbacea-Rhizobium huautlense nodulation in flooded soils and comparative characterization of S. herbaces nodulating rhizobia in different environments, Microbial Ecology, 41:25-32, 2000.
Martirani, L. et al., Purification and partial characterization of bacillocin 490, a novel bacteriocin produced by a thermophilic train of Bacillus licheniformis, Microbial Cell Factories, 1:1-5, 2002.
Masson, L. et al, Kinetics of Bacillus thuringiensis toxin binding with brush border membrane vesicles from susceptible and resistant larvae of Plutella xylostella, Journal of Biology and Chemistry, 20:11887-11896, 1995.
Mathesius, U. et al., Extensive and specific responses of a eukaryote to bacterial quorum-sensing signals, Proceedings of the National Academy of Sciences, 100:1444-1449, 2003.
Maurhofer M. et al., Influence of enhanced antibiotic production in Pseudomonas fluorescens strain CHAO on its disease suppressive capacity, Phytopathol, 82:190-195, 1992.
Mayr-Harting, A. et al., Methods for studying bacteriocins, Methods in Microbiology, 7A:315-422, 1972.
McAuliffe, O. et al., Lacticin 3147, a broad-spectrum bacteriocin which selectively dissipates the membrane potential, Applied and Environment Microbiology, 64:439-445, 1998.
Mendelsohn, M. et al., Are Bt crops safe?, Nature Biotechnology, 21:1003-1009, 2003.
Mendez-Lopez, I. et al., Bacillus thuringiensis serovar israelensis is highly toxic to the coffee berry borer, Hypothenemus hampei Ferr. (Coleoptera: Scolytidae), FEMS Microbiology Letters, 226:73-77, 2003.
Messner, P. et al., Similarity of “core” structures in two different glycans of tyrosine-linked eubacterial S-layer glycoproteins, Journal of Bacteriology, 177:2188-2193, 1995.
Mignot, T. et al., Developmental switch of S-layer protein synthesis in Bacillus anthracis, Molecular Microbiology, 43:1615-1627, 2002.
Mikesell, P. et al., Evidence for plasmid-mediated toxin production in Bacillus anthracis, Infection and Immunity, 39:371-376, 1983.
Miller, K. J. et al., Cell-associated oligosaccharides of Bradyrhizobium spp., Journal of Bacteriology, 172:136-142, 1990.
Milner, R. J., History of Bacillus thuringiensis, Agricultural Ecosystems and Environment, 49:9-13, 1994.
Moayeri, M. et al., The roles of anthrax in toxin pathogenesis, Current Opinion in Microbiology, 7:19-24, 2004.
Naclerio, G. et al., Antimicrobial activity of a newly identified bacteriocin of Bacillus cereus, Applied and Environmental Microbiology, 59:4313-4316, 1993.
Nealson, K. H. et al., Bacterial bioluminescence: its control and ecological significance, Microbiological Reviews, 43:496-518, 1979.
Novotny, J. F. et al., Characterization of bacteriocins from two strains of Bacillus thermoeovorans, a thermophilic hydrocarbon-utilizing species, Applied and Environmental Microbiology, 58:2393-2396, 1992.
Nowak R. S. et al., A test of compensatory photosynthesis in the field: Implications for herbivory tolerance, Oecologia, 61:311-318, 1984.
Nowak, J., Benefits of in vitro “biotization” of plant tissue cultures with microbial inoculants, In Vitro Cellular and Developmental Biological-Plant, 34:122-130, 1998.
Ombui, J. N., Schmieger, H., Kagiko, M. M., Arimi, S. M. 1997. Bacillus cereus may produce two or more diarrhoeal enterotoxins. FEMS Microbiology Letters 149, 245-248.
Oresnick I. J. et al., Cloning and characterization of a Rhizobium leguminosarum gene encoding a bacteriocin with similarities to RTX toxins, Applied and Environment Microbiology, 65:2833-2840, 1999.
Orozco-Cardenas, M. et al., Hydrogen peroxide is generated systemically in plant leaves by wounding and systemin via the octadecanoid pathway, Proceedings of the National Academy of Sciences of USA, 96:6553-6557, 1999.
Oscariz J. C. et al., Detection and characterization of cerein 7, a new bacteriocin produced by Bacillus cereus with a broad spectrum of activity, FEMS Microbiology Letters, 178:337-341 1999.
Padilla, C. et al., Effects of the bacteriocin PsVP-10 by Pseudomonas sp. on sensitive bacterial strains, Revista Latinoamericana de Microbiologia, 44:19-23, 2002.
Paik, H. D. et al., Identification and partial characterization of tochicin, a bacteriocin produced by B. thuringiensis subsp. Tochigiensis, Journal of Industrial Microbiology Biotechnology, 19:294-298, 1997.
Palma, J. M. et al., Increased levels of peroxisomal active oxygen-related enzymes in copper-tolerant pea plants, Plant Physiology, 85:570-574, 1987.
Papagianni, M., Ribosomally synthesized peptides with antimicrobial properties: biosynthesis, structure, function and applications, Biotechnology Advances, 21:465-499, 2003.
Parret A. H. A. et al., Bacteria killing their own kind: novel bacteriocins of Pseudomonas and other γ-proteobacteria, Trends in Microbiology, 10:107-112, 2002.
Parret, A. H. A. et al., Plant lectin-like bacteriocin from a rhizosphere-colonizing Pseudomonas isolate, Journal of Bacteriology, 185:897-908, 2003.
Pattnaik, P. et al., Purification and characterization of a bacteriocin-like compound (lichenin) produced anaerobically by Bacillus licheniformis isolated from water buffalo, Journal of Applied Microbiology, 91:636-645, 2001.
Pearce, R. B. et al., Chitin and related compounds as elicitors of the lignification response in wounded wheat leaves, Physiological Plant Pathology, 20:119-123, 1982.
Penyalzer, R. et al., Iron-binding compounds from Agrobacterium spp.: biological control strain Agrobacterium rhizogenes K84 produces a hydroxamate siderophore, Applied and Environmental Microbiology, 67:654-664, 2001.
Petosa, C. R. et al., Crystal structure of the anthrax toxin protective antigen, Nature, 385:833-838, 1997.
Priest, F. G. et al., Population structure and evolution of the Bacillus cereus group, Journal of Bacteriology, 186:7959-7970, 2004.
Prithiviraj B. et al., A host-specific bacteria-to-plant signal molecule (Nod factor) enhances germination and early growth of diverse crop plants, Planta, 216:437-445, 2003.

Probanza, A. et al., Pinus pinea L. seedling growth and bacterial rhizosphere structure after inoculation with PGPR Bacillus (B. licheniformis CET 5106 and B. pumilus CECT 5105), Applied Soil Ecology, 20:75-84, 2002.
Quadri, L. E. et al., Chemical and genetic characterization of bacteriocins produced by Carnobacterium piscicola LV17B, Journal of Biology and Chemistry, 16:12204-12211, 1994.

Racchi, M. L. et al., Differential activity of caltalase and superoxide dismutase in seedlings and in vitro micropropagated oak (Quercus robur L.), Plant Cell Reports, 20:169-174, 2001.
Rai, A. N. et al., Cyanobacterium-plant symbiosis, New Phytologist, 147:449-481, 2000.
Ramos, B. et al., Alterations in the rhizobacterial community associated with European alder growth when inoculated with PGPR strain Bacillus licheniformis, Environmental and Experimental Botany, 20:61-68, 2003.
Ramos, H. C. et al., Fermentative metabolism of Bacillus subtilis: physiology and regulation of gene expression, Journal of Bacteriology, 182:3072-3080, 2000.
Raupach, S. S. et al., Mixtures of plant growth-promoting rhizobacteria enhance biological control of multiple cucumber pathogens, Phytopathology, 11:1158-1164, 1998.
Reddell, P. et al., Transmission of infective Frankia (actinomycetales) propagules in casts of the endogeic earthworm Pontoscolex corethrurus (Oligochaeta: Glossoscolecidae), Soil Biology and Biochemistry, 23:775-778, 1991.
Reva, O, N. et al., Simplified technique for identification of the aerobic spore-forming bacteria by phenotype, International Journal of Systematic and Evolutionary Microbiology, 51:1361-1371, 2001.
Riley, M. A. et al., Bacteriocins: evolution, ecology and application, Annual Review of Microbiology, 56:117-137, 2002.
Risoen, P. A. et al., Characterization of a broad range antimicrobial substance from Bacillus cereus, Journal of Applied Microbiology, 96:648-655, 2004.
Rojas, N. S. et al., Frankia and nodulation of red alder and snowbrush grown on soils from Douglas-fir forests in the H. J. Andrews experimental forest of Oregon, Applied Soil Ecology: a Section of Agriculture, Ecosystem Environment, 17:141-149, 2001.
Rolin, D. et al., Structural studies of a phosphocholine substituted β-(1,3); (1,6) macrocyclic glucan from Bradyrhizobium japonicum USDA 110, Biochimica and Biophysica Acta, 1116:215-225, 1992.
Rosa, E. A., Global climate change: background and sociological contributions, Society Nature Research, 14:491-499, 2001.
Ryan, M. P. et al., Extensive post-translational modification, including serine to D Alanine conversion in the two-component lantibiotic, lacticin 3147, Journal of Biological Biochemistry, 53:37544-37550, 1999.
Ryu, C. M. et al., Bacterial volatiles promote growth in Arabidopsis, Proceedings of the National Academy of Sciences, 100:4927-4932, 2003.
Schripsema, J. et al., Bacteriocin small of Rhizobium leguminosarum belongs to the class of N-acyl-Lhomoserine lactone molecules, known as autoinducers and as quorum-sensing co-transcription factors, Journal of Bacteriology, 178:366-371, 1996.
Schwinghamer, E. A., Properties of some bacteriocins produced by Rhizobium trifolii, Journal of Genetics Microbiology, 91:403-413, 1975.
Seldin, L. et al., Bacillus azotofixans sp. nov., a nitrogen-fixing species from Brazilian soils and grass roots, International Journal of Systematic Bacteriology, 34:451-456, 1984.
Selvadurai, E. L. et al., Production of indole—3-accetic acid analogues by strains of Bacillus cereus in relation to their influence on seedling development, Soil Biology and Biochemistry, 23:401-403, 1990.
Shida, O. et al., Proposal for two new genera, Brevibacillus gen. nov. and Aneurinibacillus gen. nov., International Journal of Systematic Bacteriology, 46:939-946, 1996.
Shishido, M. et al., Effect of plant growth promoting Bacillus strains on pine and spruce seedling growth and mycorrhizal infection, Annals of Botany, 77:433-441, 1996.
Singleton, V. L. et al., Colorimetry of total phenolics with phosphomolybdic-phosphotungstic acid reagents, American Journal of Enology and Viticulture, 16:144-158, 1965.
Sloma, A. et al., RNA synthesis during spore germination in Bacillus subtilis, Molecular Genetics, 175:113-120, 1979.
Smith, D. L. et al., Climate change and crop production: contributions, impacts and adaptations, Canadian Journal of Plant Pathology, 26:253-266, 2004.
Somssich, I. E. et al., Rapid activation by fungal elicitor of genes encoding pathogenesis-related proteins in cultured parsley cells, Proceedings of the National Academy of Science of USA, 83:2427-2430, 1986.
Souleimanov A et al., The major Nod factor of Bradyrhizobium japonicum promotes early growth of soybean and corn, Journal of Experimental Botany, 53:1929-1934, 2002.
Steidle, A. et al., Identification and characterization of an N-acylhomoserine lactone-dependent quorum-sensing system in Pseudomonas putida strain IsoF, Applied and Environmental Microbiology, 68:6371-6382, 2002.
Stenlid, G., Cytokinins as inhibitors of root growth, Physiologia Plantarum, 56:500-506, 1982.
Sturz, A. V. et al., Bacterial endophytes: potential role in developing sustainable systems of crop production, Critical Reviews in Plant Science, 19:1-30, 2000.
Tagg, J. R. et al., Bacteriocins of Gram-positive bacteria, Bacteriological Reviews, 40:722-756, 1976.
Thelen G. C. et al., Insect herbivory stimulates allelopathic exudation by an invasive plant and the suppression of natives, Ecological Letters, 8:209-217, 2005.
Thrane, C. et al., A note: direct microscopy of Bacillus endospore germination in soil microcosms, Journal of Applied Microbiology, 89:595-598, 2000.
Ticknor, L. O. et al., Fluorescent amplified fragment length polymorphism analysis of Norwegian Bacillus cereus and Bacillus thuringiensis soil isolates, Applied and Environmental Microbiology, 67:4863-4873, 2001.
Timmusk, S. et al., Cytokinin production by Paenibacillus polymyxa, Soil Biology and Biochemistry, 31:1847-1852, 1999.
Torkar, K. G. et al., Partial characterization of bacteriocins produced by Bacillus cereus isolates from milk and milk products, Food Technology and Biotechnology, 41:121-129, 2003.
Trumble J. T. et al., Plant compensation for arthropod herbivory, Annual Review of Entomology, 38:93-119, 1993.
Vander, P. et al., Comparison of the ability of partially N-acetylated chitosans and chitooligosaccharides to elicit resistance reactions in wheat leaves, Plant Physiology, 118:1353-1359, 1998.
Vaughan-Martini, A. et al., Use of conventional taxonomic electrophoretic karyotyping and DNA-DNA hybridization of the classification of fermentative apiculate yeasts, International Journal of Systematic Evolution and Microbiology, 50:1665-1672, 2000.
Venter A. R. et al., Analysis of the genetic region encoding a novel bacteriocin from Rhizobium leguminosarum viciae strain 306, Canadian Journal of Microbiology, 47:495-502, 2001.
Vessey, K. V., Plant growth promoting rhizobacteria as biofertlizers, Plant and Soil, 255:571-586, 2003.
Vincent J. M., A manual for the practical study of root nodule bacteria, Blackwell Scientific Publication, Oxford, UK, 1970.
von Tersch, M. A. et al., Bacteriocin from Bacillus megatarium ATCC19213: comparative studies of megacin A-216, Journal of Bacteriology, 155:872-877, 1983.
Vonderwell, J. D. et al, Influence of two plant growth promoting rhizobacteria on loblolly pine root respiration and IAA activity, Forest Science, 47:197-202, 2000.
Wang, B. et al., Use of defined mutants to assess the role of the Campylobacter rectus S-layer in bacterium-epithelial cell interactions, Infection and Immunity, 68:1465-1473, 2000.
Weidhase, R. A. et al., Degradation of ribulose-1,5-bisphosphate carboxylase and chlorophyll in senescing barley leaf segments triggered by jasmonic acid methylester, and counteraction by cytokinin, Physiolgia Plantarium, 69:161-166, 1987.
Welkos, S. et al., The role of antibodies to Bacillus anthracis and anthrax toxin component in inhibiting the early stages of infection by anthrax spores, Microbiology, 147:1677-1685, 2001.
Wilkinson, A. et al., N-Acyl-Homoserin lactone inhibition of rhizobial growth is mediated by two quorum-sensing genes that regulate plasmid transfer, Journal of Bacteriology, 16:4510-4519, 2002.
Wilson R. A. et al., Bacteriocin production and resistance in a field population of Rhizobium leguminosarum biovar viciae, Soil Biology and Biochemistry, 30:413-417, 1998.
Wu, C. W. et al., Purification and characterization of bacteriocin from Pediococcus pentosaceus ACCEL, Journal of Agricultural Food Chemistry, 5:1146-1151, 2004.
Zakharova, E. et al., Biosynthesis of indole-3-acetic acid in Azospirillum brasilense: insights from quantum chemistry, European Journal of Biochemistry, 259:572-576, 1999.
Zhang F. et al., Effects of low root zone temperature on the early stages of symbiosis establishment between soybean [Glycine max. (L.) Merr] and Bradyrhizobium japonicum, Journal of Experimental Botany, 279:1467-1473, 1994.
Zhang F. et al., Preincubation of Bradyrhizobium japonicum with genistein accelerates nodule development of soybean at suboptimal root zone temperatures, Plant Physiology, 108:961-968, 1995.
Zheng, G. et al., Isolation, partial purification and characterization of a bacteriocin produced by a newly isolated Bacillus subtilis strain, Letters in Applied Microbiology, 28:363-367, 1999.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

As can be understood by one skilled in the art, many modifications to the exemplary embodiments described herein are possible. The invention, rather, is intended to encompass all such modification within its scope, as defined by the claims.

1. A method for promoting plant growth and/or disease resistance comprising applying a purified polypeptide that is a bacteriocin and that possesses plant growth and/or disease resistance promoting activity to a plant or plant seed, or to the growing environment thereof.

2. The method according to claim 1, wherein said purified polypeptide is obtained from or obtainable from a plant growth promoting rhizobacteria (PGPR).

3. (canceled)

4. The method according to claim 2, wherein said PGPR is a PGPR of the genus Bacillus, Pseudomonas, Rhizobium, or Bradyrhizobium.

5. The method according to claim 2, wherein said PGPR is of the species Bacillus thuringiensis.

6. The method according to claim 2, wherein said PGPR has the identifying characteristics of Bacillus thuringiensis strain NEB17 (deposited at the International Depositary Authority of Canada (IDAC) on Mar. 27, 2003 under Accession No. 270303-02), B. thuringiensis strain BUPM4 or B. cereus strain UW85 (ATCC 53522).

7. The method according to claim 2, wherein said PGPR is Bacillus thuringiensis strain NEB17 (IDAC 270303-02), B. thuringiensis strain BUPM4 or B. cereus strain UW85 (ATCC 53522).

8. (canceled)

9. (canceled)

10. (canceled)

11. (canceled)

12. (canceled)

13. The method according to claim 1, said polypeptide being selected from the group consisting of:
(a) a polypeptide comprising the partial amino acid sequence WTCWSCLVCAACSVELL (SEQ ID NO: 1);
(b) a polypeptide possessing the bacteriocin and plant growth and/or disease resistance promoting activities of the polypeptide of (a), and which comprises a sequence of 17 contiguous amino acids possessing at least 70% sequence identity to SEQ ID NO: 1; and
(c) a polypeptide which is a fragment of the polypeptide of (a) or (b), said fragment possessing the bacteriocin and plant growth and/or disease promoting activities of the polypeptide of (a).

14. The method according to claim 1, wherein said polypeptide:
(a) comprises the partial amino acid sequence WTCWSCLVCAACSVELL (SEQ ID NO: 1);
(b) has a molecular weight in the range of about 3100 to about 3200 Da;
(c) is obtainable from Bacillus thuringiensis strain NEB17 (IDAC 270303-02);
(d) maintains bactericidal and/or bacteristatic activity after exposure to 100° C. for 15 minutes; and
(e) maintains bactericidal and/or bacteristatic activity after treatment α-amylase or catalase, and exhibits loss of activity after treatment with proteinase K or protease.

15. (canceled)

16. The method according to claim 1, wherein said plant is a legume, corn or tomato plant.

17. The method according to claim 16, wherein said plant is a soybean.

18. The method according to claim 1, wherein said plant exhibits an increase in one or more of: (a) nodulation; (b) leaf area; (c) seed germination; (d) leaf greenness; (e) photosynthesis; (f) accumulated dry weight; (g) phenylalanine ammonia lyase (PAL); (h) tyrosine ammonia lyase (TAL); (i) peroxidase (POD); (j) catalase (CAT); (k) superoxidase dismutase (SOD); or (l) total phenolic compounds, relative to a control plant.

19. A purified polypeptide that is a bacteriocin and that possesses plant growth and/or disease resistance promoting activity, said polypeptide being selected from the group consisting of:
(a) a polypeptide comprising the partial amino acid sequence

| | |
|---|---|
| WTCWSCLVCAACSVELL; | (SEQ ID NO: 1) |

(b) a polypeptide possessing the bacteriocin and plant growth and/or disease resistance promoting activities of the polypeptide of (a), and which comprises a sequence of 17 contiguous amino acids possessing at least 70% sequence identity to SEQ ID NO: 1; and
(c) a polypeptide which is a fragment of the polypeptide of (a) or (b), said fragment possessing the bacteriocin and plant growth and/or disease resistance promoting activities of the polypeptide of (a).

20. The polypeptide according to claim 19, said polypeptide having one or more of the following properties:
(a) bactericidal and/or bacteristatic activity against one or more strains of Bacillus thuringiensis, Bacillus cereus or Escherichia coli;
(b) retention of bactericidal and/or bacteristatic activity after exposure to 100° C. for 15 minutes;
(c) retention of bactericidal and/or bacteristatic activity after treatment with α-amylase or loss of activity after treatment with proteinase K or protease;
(d) bactericidal and/or bacteristatic activity against human-, animal-, or food-borne pathogens;
(e) a molecular weight in the range of about 3100 to about 3200 Da;
(f) is obtained from or is obtainable from a plant growth promoting rhizobacteria (PGPR) having the identifying characteristics of Bacillus thuringiensis strain NEB17 (deposited at the International Depositary Authority of Canada (IDAC) on Mar. 27, 2003 under Accession No. 270303-02).

21. The purified polypeptide according to claim 20, wherein said polypeptide:
(a) comprises the partial amino acid sequence DWTCWSCLVVAACSVELL;
(b) has a molecular weight in the range of about 3100 to about 3200 Da;
(c) is obtained from or is obtainable from Bacillus thuringiensis strain NEB17 (IDAC 270303-02);
(d) maintains bactericidal and/or bacteristatic activity after exposure to 100° C. for 15 minutes; and
(e) maintains bactericidal and/or bacteristatic activity after treatment α-amylase or catalase, and exhibits loss of activity after treatment with proteinase K or protease.

22. (canceled)

23. (canceled)

24. An isolated polynucleotide encoding the polypeptide according to claim 19, or the complement thereto.

25. (canceled)

26. (canceled)

27. A host cell comprising the polynucleotide according to claim 24.

28. (canceled)

29. A plant growth and/or disease resistance promoting composition comprising a purified polypeptide as defined in claim 1 and a carrier or diluent.

30. A plant seed treated with the plant growth and/or disease resistance promoting composition according to claim 29.

31. (canceled)

32. A method for obtaining a polypeptide as defined in claim 1, comprising:
(a) providing a polypeptide;
(b) determining whether said polypeptide promotes plant growth and/or disease resistance; and
(c) determining whether said polypeptide has bactericidal and/or bacteristatic properties.

33. (canceled)

34. A method for obtaining the polypeptide as defined in claim 1, comprising:
(a) providing a bacteriocin; and
(b) determining whether said bacteriocin has plant growth and/or disease resistance promoting properties.

35. (canceled)
